SEQUENCE LISTING (35 sequences)

SEQ ID NO: 1 - DNA, 1029 bp, Enterobacteria phage P1
atgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgat

SEQ ID NO: 2 - DNA, 34 bp, Enterobacteria phage P1
ataacttcgtatagcatacattatacgaagttat

SEQ ID NO: 3 - DNA, 34 bp, artificial sequence
  Description of Artificial Sequence lox2272 sequence
ataacttcgtataggataccttatacgaagttat

SEQ ID NO: 4 - DNA, 60 bp, artificial sequence
  Description of Artificial Sequence Gsk-3a short hairpin oligonucleotide sense
ctcattcggagtagtataccgaagcttgggtatactactccgaatgagcttttttggaaa

SEQ ID NO: 5 - DNA, 66 bp, artificial sequence
  Description of Artificial Sequence Gsk-3a short hairpin oligonucleotide antisense
cgctcattcggagtagtataccgaagcttgggtatactactccgaatgagcttttttggaaagatc

SEQ ID NO: 6 - DNA, 60 bp, artificial sequence
  Description of Artificial Sequence Gsk-3 beta short hairpin oligonucleotide sense
ctgtgtgttggctgaattgtgaagcttgacaattcagccaacacacagcttttttggaaa

SEQ ID NO: 7 - DNA, 66 bp, artificial sequence
  Description of Artificial Sequence Gsk-3 beta short hairpin oligonucleotide antisense
cgctgtgtgttggctgaattgtgaagcttgacaattcagccaacacacagcttttttggaaagatc

SEQ ID NO: 8 - DNA, 3278 bp, artificial sequence
  Description of Artificial Sequence pbsU6 vector
ataaaacctgcaggcatgcaagcgatcgcggggccgcccccttcaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggcccattcctcctcggatccaagggtgggcgcgccaaggcccgcggggccactagttctagagcggccccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggaaacatgcatgaagttcctattccgaagttcctattctctagaaagtataggaacttc

SEQ ID NO: 9 - DNA, 883 bp, artificial sequence
  Description of Artificial Sequence stop cassette loxP-stop-loxP
gagtcgactgataacttcgtatagcatacattatacgaagttatggatccagcttggtagcgcggtgtattatactttttggaaagaattcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcaattctctagcgcctgatgcggtattttctccttacgcatctgtgcggtatttcacaccgcatattttttggatccataacttcgtatagcatacattatacgaagttatgactggactc

SEQ ID NO: 10 - DNA, 883 bp, artificial sequence
  Description of Artificial Sequence stop cassette lox2272-stop-lox2272
gagtcgactgataacttcgtataggataccttatacgaagttatggatccagcttggtagcgcggtgtattatactttttggaaagaattcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcaattctctagcgcctgatgcggtattttctccttacgcatctgtgcggtatttcacaccgcatattttttggatccataacttcgtataggataccttatacgaagttatgactggactc

SEQ ID NO: 11 - DNA, 4190 bp, artificial sequence
  Description of Artificial Sequence pbsU6-shGsk-3a-flox2
ataaaacctgcaggcatgcaagcgatcgcggggccgcccccttcaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgctcattcggagtagtataccgaagctataacttcgtataggataccttatacgaagttatggatccagcttggtagcgcggtgtattatactttttggaaagaattcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcaattctctagcgcctgatgcggtattttctccttacgcatctgtgcggtatttcacaccgcatattttttggatccataacttcgtataggataccttatacgaagttatagcttgggtatactactccgaatgagcttttttggaaagatccaagggtgggcgcgccaaggcccgcggggccactagttctagagcggccccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggaaacatgcatgaagttcctattccgaagttcctattctctagaaagtataggaacttc

SEQ ID NO: 12 - DNA, 4190 bp, artificial sequence
  Description of Artificial Sequence pbsU6-shGsk-3 beta-flox
ataaaacctgcaggcatgcaagcgatcgcggggccgcccccttcaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgctgtgtgttggctgaattgtgaagctataacttcgtatagcatacattatacgaagttatggatccagcttggtagcgcggtgtattatactttttggaaagaattcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcaattctctagcgcctgatgcggtattttctccttacgcatctgtgcggtatttcacaccgcatattttttggatccataacttcgtatagcatacattatacgaagttatagcttgacaattcagccaacacacagcttttttggaaagatccaagggtgggcgcgccaaggcccgcggggccactagttctagagcggccccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggaaacatgcatgaagttcctattccgaagttcctattctctagaaagtataggaacttc

SEQ ID NO: 13 - DNA, 4380 bp, artificial sequence
  Description of Artificial Sequence pRMCE-II
caattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctgggtaccgggccccccctcgacgcgccgtcgacgtttgatccccgcggtgcgggtgccagggcgtgcccttgggctccccgggcgcgtactccacggatcaaacgcgctgttctcctcttcctcatctccgggcctttcgacctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcaattctctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgaaaacatgcatgaagttcctattccgaagttcctattctctagaaagtataggaacttcataaaacctgcaggcatgcaagcgattaactttaaataattggcattatttaaagttagcgatcgcggccggcccgcggggcctaactttaaataattggcattatttaaagttagcggggccactagttctagagcgatccccgcggtgcgggtgccagggcgtgcccttgggctccccgggcgcgtactccacggatcgccaccgcggtggagctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacg

SEQ ID NO: 14 - DNA, 5593 bp, artificial sequence
  Description of Artificial Sequence pRMCE-II-U6-shGsk-3 beta-flox
attaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctgggtaccgggccccccctcgacgcgccgtcgacgtttgatccccgcggtgcgggtgccagggcgtgcccttgggctccccgggcgcgtactccacggatcaaacgcgctgttctcctcttcctcatctccgggcctttcgacctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcaattctctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgaaaacatgcatgaagttcctattccgaagttcctattctctagaaagtataggaacttcataaaacctgcaggcatgcaagcgattaactttaaataattggcattatttaaagttagcgatcgcggggccgcccccttcaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgctgtgtgttggctgaattgtgaagctataacttcgtatagcatacattatacgaagttatggatccagcttggtagcgcggtgtattatactttttggaaagaattcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcaattctctagcgcctgatgcggtattttctccttacgcatctgtgcggtatttcacaccgcatattttttggatccataacttcgtatagcatacattatacgaagttatagcttgacaattcagccaacacacagcttttttggaaagatccaagggtgggcgcgccaaggcccgcggggcctaactttaaataattggcattatttaaagttagcggggccactagttctagagcgatccccgcggtgcgggtgccagggcgtgcccttgggctccccgggcgcgtactccacggatcgccaccgcggtggagctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgca

SEQ ID NO: 15 - DNA, 6879 bp, artificial sequence
  Description of Artificial Sequence pRMCE-II-U6-shGsk-3 beta-flox-U6-shGsk-3a-flox2
caattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctgggtaccgggccccccctcgacgcgccgtcgacgtttgatccccgcggtgcgggtgccagggcgtgcccttgggctccccgggcgcgtactccacggatcaaacgcgctgttctcctcttcctcatctccgggcctttcgacctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcaattctctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgaaaacatgcatgaagttcctattccgaagttcctattctctagaaagtataggaacttcataaaacctgcaggcatgcaagcgattaactttaaataattggcattatttaaagttagcgatcgcggggccgcccccttcaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgctgtgtgttggctgaattgtgaagctataacttcgtatagcatacattatacgaagttatggatccagcttggtagcgcggtgtattatactttttggaaagaattcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcaattctctagcgcctgatgcggtattttctccttacgcatctgtgcggtatttcacaccgcatattttttggatccataacttcgtatagcatacattatacgaagttatagcttgacaattcagccaacacacagcttttttggaaagatccaagggtgggcgcgccaaggcccgcggggcctaactttaaataattggcattatttaaagttagcggggccactagaaagtataggaacttcataaaacctgcaggcatgcaagcgatcgcggggccgcccccttcaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgctcattcggagtagtataccgaagctataacttcgtataggataccttatacgaagttatggatccagcttggtagcgcggtgtattatactttttggaaagaattcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcaattctctagcgcctgatgcggtattttctccttacgcatctgtgcggtatttcacaccgcatattttttggatccataacttcgtataggataccttatacgaagttatagcttgggtatactactccgaatgagcttttttggaaagatccaagggtgggcgcgccaaggcccgcggggccactagttctagttctagagcgatccccgcggtgcgggtgccagggcgtgcccttgggctccccgggcgcgtactccacggatcgccaccgcggtggagctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacg

SEQ ID NO: 16 - DNA, 6576 bp, artificial sequence
  Description of Artificial Sequence pCAG-C31Int-bpA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccgggggcgcgccggatctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagatccttaattaagtctagaccgatatgacacaaggggttgtgaccggggtggacacgtacgcgggtgcttacgaccgtcagtcgcgcgagcgcgagaattcgagcgcagcaagcccagcgacacagcgtagcgccaacgaagacaaggcggccgaccttcagcgcgaagtcgagcgcgacgggggccggttcaggttcgtcgggcatttcagcgaagcgccgggcacgtcggcgttcgggacggcggagcgcccggagttcgaacgcatcctgaacgaatgccgcgccgggcggctcaacatgatcattgtctatgacgtgtcgcgcttctcgcgcctgaaggtcatggacgcgattccgattgtctcggaattgctcgccctgggcgtgacgattgtttccactcaggaaggcgtcttccggcagggaaacgtcatggacctgattcacctgattatgcggctcgacgcgtcgcacaaagaatcttcgctgaagtcggcgaagattctcgacacgaagaaccttcagcgcgaattgggcgggtacgtcggcgggaaggcgccttacggcttcgagcttgtttcggagacgaaggagatcacgcgcaacggccgaatggtcaatgtcgtcatcaacaagcttgcgcactcgaccactccccttaccggacccttcgagttcgagcccgacgtaatccggtggtggtggcgtgagatcaagacgcacaaacaccttcccttcaagccgggcagtcaagccgccattcacccgggcagcatcacggggctttgtaagcgcatggacgctgacgccgtgccgacccggggcgagacgattgggaagaagaccgcttcaagcgcctgggacccggcaaccgttatgcgaatccttcgggacccgcgtattgcgggcttcgccgctgaggtgatctacaagaagaagccggacggcacgccgaccacgaagattgagggttaccgcattcagcgcgacccgatcacgctccggccggtcgagcttgattgcggaccgatcatcgagcccgctgagtggtatgagcttcaggcgtggttggacggcagggggcgcggcaaggggctttcccgggggcaagccattctgtccgccatggacaagctgtactgcgagtgtggcgccgtcatgacttcgaagcgcggggaagaatcgatcaaggactcttaccgctgccgtcgccggaaggtggtcgacccgtccgcacctgggcagcacgaaggcacgtgcaacgtcagcatggcggcactcgacaagttcgttgcggaacgcatcttcaacaagatcaggcacgccgaaggcgacgaagagacgttggcgcttctgtgggaagccgcccgacgcttcggcaagctcactgaggcgcctgagaagagcggcgaacgggcgaaccttgttgcggagcgcgccgacgccctgaacgcccttgaagagctgtacgaagaccgcgcggcaggcgcgtacgacggacccgttggcaggaagcacttccggaagcaacaggcagcgctgacgctccggcagcaaggggcggaagagcggcttgccgaacttgaagccgccgaagccccgaagcttccccttgaccaatggttccccgaagacgccgacgctgacccgaccggccctaagtcgtggtgggggcgcgcgtcagtagacgacaagcgcgtgttcgtcgggctcttcgtagacaagatcgttgtcacgaagtcgactacgggcagggggcagggaacgcccatcgagaagcgcgcttcgatcacgtgggcgaagccgccgaccgacgacgacgaagacgacgcccaggacggcacggaagacgtagcggcgcctaagaagaagaggaaggtttagtctagagtcgactgtttctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgagatccactagttctagcctcgaggctagagcggccaaacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 17 - DNA, 6081 bp, artificial sequence
  Description of Artificial Sequence pCAG-Cre-bpA
gggtaccgggccccccctcgaggtcgacggtatcgataagcttgatatcgaattcgagctcggtacccgggggcgcgccggatctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagatccttaattaagtctagagtcgactgtttaaacctgcagctcgaggtcgaccatgcccaagaagaagaggaaggtgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattagccattaacgcgtaaatgattgcagatccactagttctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgagatccactagttctagcctcgaggctagagcggccgccaccgcggtggagctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagct

SEQ ID NO: 18 - DNA, 34 bp, artificial sequence
  Description of Artificial Sequence Lox 5171
ataacttcgtataatgtgtactatacgaagttat

SEQ ID NO: 19 - DNA, 34 bp, artificial sequence
  Description of Artificial Sequence Lox 5271
ataacttcgtataatgtttactatacgaagttat

SEQ ID NO: 20 - DNA, 34 bp, artificial sequence
  Description of Artificial Sequence Lox 5371
ataacttcgtataatgtctactatacgaagttat

SEQ ID NO: 21 - DNA, 34 bp, artificial sequence
  Description of Artificial Sequence Lox 5172
ataacttcgtataatgtgtcctatacgaagttat

SEQ ID NO: 22 - DNA, 34 bp, artificial sequence
  Description of Artificial Sequence Lox 5272
ataacttcgtataatgtttcctatacgaagttat

SEQ ID NO: 23 - DNA, 34 bp, artificial sequence
  Description of Artificial Sequence Lox 5372
ataacttcgtataatgtctcctatacgaagttat

SEQ ID NO: 24 - DNA, 34 bp, artificial sequence
  Description of Artificial Sequence m2
ataacttcgtataagaaaccatatacgaagttat

SEQ ID NO: 25 - DNA, 34 bp, artificial sequence
  Description of Artificial Sequence m3
ataacttcgtatataataccatatacgaagttat

SEQ ID NO: 26 - DNA, 34 bp, artificial sequence
  Description of Artificial Sequence m7
ataacttcgtataagatagaatatacgaagttat

SEQ ID NO: 27 - DNA, 34 bp, artificial sequence
  Description of Artificial Sequence m11
ataacttcgtatacgataccatatacgaagttat

SEQ ID NO: 28 - DNA, 23 bp, artificial sequence
  Description of Artificial Sequence pgk-F
cacgcttcaaaagcgcacgtctg

SEQ ID NO: 29 - DNA, 25 bp, artificial sequence
  Description of Artificial Sequence neo-R
gttgtgcccagtcatagccgaatag

SEQ ID NO: 30 - DNA, 28 bp, artificial sequence
  Description of Artificial Sequence Hyg-1
gaagaatctcgtgctttcagcttcgatg

SEQ ID NO: 31 - DNA, 25 bp, artificial sequence
  Description of Artificial Sequence Hyg-2
aatgaccgctgttatgcggccattg

SEQ ID NO: 32 - DNA, 20 bp, artificial sequence
  Description of Artificial Sequence Rosa-5′
cgtgttcgtgcaagttgagt

SEQ ID NO: 33 - DNA, 19 bp, artificial sequence
  Description of Artificial Sequence Rosa-3′
actcccgcccatcttctag

SEQ ID NO: 34 - DNA, 19 bp, artificial sequence
  Description of Artificial Sequence Probe-F
aaggatactggggcatacg

SEQ ID NO: 35 - DNA, 23 bp, artificial sequence
  Description of Artificial Sequence Probe-R
cttctcagctacctttacacacc

(19) United States
(12) Patent Application Publication
(10) Pub. No.: US 2010/0299771 A1
(43) Pub. Date: Nov. 25, 2010

(54) MEANS AND METHODS FOR shRNA MEDIATED CONDITIONAL KNOCKDOWN OF GENES

(75) Inventors: Ralf Kühn, Freising (DE); Wolfgang Wurst, Munchen (DE); Patricia Steuber-Buchberger, Scheyern (DE)

Correspondence Address: PILLSBURY WINTHROP SHAW PITTMAN LLP, ATTENTION: DOCKETING DEPARTMENT, P.O BOX 10500, McLean, VA, 22102, US

(73) Assignee: HELMHOLTZ ZENTRUM MUNCHEN- DEUTSCHES FORSCHUNGZENTRUM FUR GESUNDHEIT UND UMWELT(GMBH), 85764 Neuherberg (DE)

(21) Appl. No.: 12/678,645

(22) PCT Filed: Sep. 17, 2008
(86) PCT No.: PCT/EP08/07779

§ 371 Date: Aug. 5, 2010

Related U.S. Application Data
(60) Provisional application No. 60/973,055, filed on Sep. 17, 2007.

(51) Int. Cl. A01K 67/027; C12N 15/63; A01K 67/00; C12N 5/10; C12N 15/09; C12Q 1/68

(52) U.S. Cl. 800/18; 435/320.1; 800/22; 800/13; 800/14; 435/325; 435/455; 435/6

(57) ABSTRACT

The present invention relates to a combination of DNA segments comprising: (a) a first segment comprising in 5′ to 3′ or 3′ to 5′ order: (aa) a promoter; (ab) a first DNA sequence comprising: (i) a DNA sequence giving rise upon transcription to the sense strand of an shRNA molecule; (ii) a transcriptional stop element which is flanked by a first type of recombinase recognition sequences; and (iii) a DNA sequence giving rise upon transcription to the antisense strand of an shRNA molecule; (b) a second segment comprising in 5′ to 3′ or 3′ to 5′ order: (ba) a promoter; (bb) a second DNA sequence comprising: (i) a DNA sequence giving rise upon transcription to the sense strand of an shRNA molecule; (ii) a transcriptional stop element which is flanked by a second type of recombinase recognition sequences; and (iii) a DNA sequence giving rise upon transcription to the antisense strand of an shRNA molecule; wherein (i) said first type of recombinase recognition sequences are recognized and recombined by a recombinase but not recombined with said second type of recombinase recognition sequences; (ii) said second type of recombinase recognition sequences are recognized and recombined by the recombinase of (i) but not recombined with said first type of recombinase recognition sequences; and (iii) said DNA sequence of (ab) and (bb) is expressed under the control of said promoters of (aa) and (ba) upon removal of said transcriptional stop elements of (ab) and (bb) by the activity of a recombinase, resulting in transcription of said shRNA molecule in a cell. Further, the invention relates to a genetically engineered non-human animal and a method to produce said transgenic non-human animal. Also, the invention relates to a cell genetically engineered with the DNA molecule of the invention and a method of simultaneously knocking down two genes in a cell. Furthermore, envisaged is a method of identifying a combination of two target genes as a potential drug target and the use of the DNA molecule of the invention for the preparation of a composition for gene therapy.

The present invention relates to a combination of DNA segments comprising: (a) a first segment comprising in 5′ to 3′ or 3′ to 5′ order: (aa) a promoter; (ab) a first DNA sequence comprising: (i) a DNA sequence giving rise upon transcription to the sense strand of an shRNA molecule; (ii) a transcriptional stop element which is flanked by a first type of recombinase recognition sequences; and (iii) a DNA sequence giving rise upon transcription to the antisense strand of an shRNA molecule; (b) a second segment comprising in 5′ to 3′ or 3′ to 5′ order: (ba) a promoter; (bb) a second DNA sequence comprising: (i) a DNA sequence giving rise upon transcription to the sense strand of an shRNA molecule; (ii) a transcriptional stop element which is flanked by a second type of recombinase recognition sequences; and (iii) a DNA sequence giving rise upon transcription to the antisense strand of an shRNA molecule; wherein (i) said first type of recombinase recognition sequences are recognized and recombined by a recombinase but not recombined with said second type of recombinase recognition sequences; (ii) said second type of recombinase recognition sequences are recognized and recombined by the recombinase of (i) but not recombined with said first type of recombinase recognition sequences; and (iii) said DNA sequence of (ab) and (bb) is expressed under the control of said promoters of (aa) and (ba) upon removal of said transcriptional stop elements of (ab) and (bb) by the activity of a recombinase, resulting in transcription of said shRNA molecule in a cell. Further, the invention relates to a genetically engineered non-human animal and a method to produce said transgenic non-human animal. Also, the invention relates to a cell genetically engineered with the DNA molecule of the invention and a method of simultaneously knocking down two genes in a cell. Furthermore, envisaged is a method of identifying a combination of two target genes as a potential drug target and the use of the DNA molecule of the invention for the preparation of a composition for gene therapy.

Several documents are cited throughout the text of this specification. The disclosure content of the documents cited herein (including manufacturer's specifications, instructions, etc.) is herewith incorporated by reference.

RNA interference (RNAi) is a mechanism for RNA-guided regulation of gene expression and is conserved in most eukaryotic organisms. The RNAi pathway is thought to have evolved as a form of innate immunity against viruses and also plays a major role in regulating development and genome maintenance. In research, RNAi has become an extremely useful genetic tool to study gene function in mammalian cells. The discovery that short interfering (si)RNAs avoid an interferon response and the global shutdown of translation has enabled the wide use of transient gene silencing in cultured cells and specific tissues of mice upon local administration (Lieberman et al., (2003), Trends Mol. Med., 9, 397-403). To elicit permanent gene silencing, short hairpin (sh)RNA expression vectors can be used. These vectors consist of an RNA polymerase III promoter producing short RNA fragments, which form hairpin structures. Subsequently, the shRNAs are processed by the RNAi machinery resulting in siRNAs which subsequently mediate sequence-specific gene silencing (Dykxhoorn et al., (2003), Nat. Rev. Mol. Cell Biol., 4, 457-467).

The shRNA vectors have also been successfully used to produce an all-over knockdown phenotype similar to conventional knockout mice by creating mice transgenic for an shRNA vector (Kunath et al., (2003), Nat. Biotechnol., 21, 559-561). This method has proved to be useful in solving the problem of embryonic lethality arising from inter alia targeting of genes which are somehow involved in the developmental phase of the embryo. Conditional vectors allow for regulated expression of shRNA molecules and thus expression can be turned on leading to knockdown of the target gene in a tissue-specific and/or time dependent manner. Regulation can be achieved by using an inducing compound such as, for example, doxycycline which acts on artificial regulatory sequences in the polymerase III promoter (Chen et al., (2003), Cancer Res., 63, 4801-4804) or by lifting a blockade of transcription. Transcriptional stop elements can block transcription and can be excised using the Cre/IoxP approach to allow for time- and/or tissue-specific knock down of a gene. Various vector designs for Cre/IoxP mediated RNAi have been described (Kasim et al., (2004), Nucleic Acids Res., 32, e66; Tiscomia et al., (2004), Proc. Natl Acad Sci. USA, 101, 7347-7351), nevertheless, there is a steady demand for improved gene targeting and knockdown methods for use in a variety of fields.

The technical problem underlying the present invention was to identify alternative and/or improved means and methods that allow for gene knockdown.

The solution to this technical problem is achieved by providing the embodiments characterized in the claims.

Accordingly, the present invention relates in a first embodiment to a combination of DNA segments comprising:
(a) a first segment comprising in 5′ to 3′ or 3′ to 5′ order:
   (aa) a promoter;
   (ab) a first DNA sequence comprising:
      (i) a DNA sequence giving rise upon transcription to the sense strand of an shRNA molecule;
      (ii) a transcriptional stop element which is flanked by a first type of recombinase recognition sequences; and
      (iii) a DNA sequence giving rise upon transcription to the antisense strand of an shRNA molecule;
(b) a second segment comprising in 5′ to 3′ or 3′ to 5′ order:
   (ba) a promoter I;
   (bb) a second DNA sequence comprising:
      (i) a DNA sequence giving rise upon transcription to the sense strand of an shRNA molecule;
      (ii) a transcriptional stop element which is flanked by a second type of recombinase recognition sequences; and
      (iii) a DNA sequence giving rise upon transcription to the antisense strand of an shRNA molecule;
wherein
(i) said first type of recombinase recognition sequences are recognized and recombined by a recombinase but not recombined with said second type of recombinase recognition sequences;
(ii) said second type of recombinase recognition sequences are recognized and recombined by the recombinase of (i) but not recombined with said first type of recombinase recognition sequences; and
(iii) said DNA sequence of (ab) and (bb) is expressed under the control of said promoters of (aa) and (ba) upon removal of said transcriptional stop elements of (ab) and (bb) by the activity of a recombinase, resulting in transcription of said shRNA molecule in a cell.

The term “promoter” relates to promoters which are functional in a cell and mediate the expression of the shRNA molecule within said cell. The structure and function of prokaryotic and eukaryotic promoters are well-known to the person skilled in the art and described, for example, in “Molecular Cell Biology”, Lodish et al. (eds), W.H. Freeman&Co, New York. The promoters may be RNA polymerase II or III dependent and constitutively active or inducible, ubiquitous or gene-/tissue-specific. Further, the promoters may contain artificially introduced sequences to modify their regulatory capacity, such as, for example, enhancers, silencers, insulators, specific transcription factor binding sites or specific operator sequences like to, tet, Gal4, lac conferring inducibility to said promoter. Preferred cells are eukaryotic cells. Accordingly, preferred are promoters which are functional in eukaryotic cells.

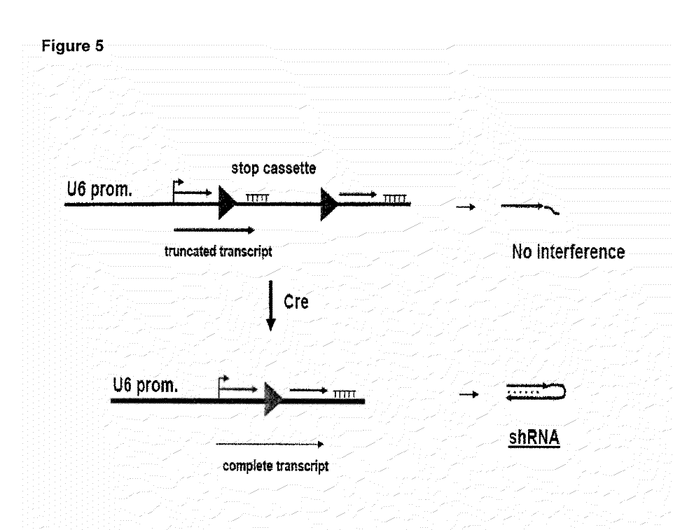

The terms “sense” and “antisense strand of an shRNA molecule” relates to the RNA strands which due to their complementary RNA sequence undergo basepairing and form the characteristic double-strand RNA segment of an shRNA molecule which is responsible for mediating RNA interference. The shRNA molecule generated in accordance with the invention is composed of a double stranded segment and further a single stranded segment commonly referred to as the “stem loop” or “hairpin” structure (cf. FIG. 5). Said structure is the result of the basepairing of the complementary RNA sequences of the sense and antisense strand which flank a stretch of non-complementary nucleotides. The stem loop also comprises a Iox sequence wherein said Iox sequence is the result of the excision of the transcriptional stop element and the subsequent ligation of the DNA strand ends encoding the shRNA molecule.

The term “a transcriptional stop element” as used in accordance with the present invention relates to a transcriptional stop element which is located within the region of the DNA sequence that after excision constitutes the region of the shRNA molecule that does not contain base pairs complementary to each other thus not forming an RNA double strand, commonly referred to as the “loop”. A transcriptional stop element may be any element which due to its nucleotide sequence leads to the arrest of transcription of functional, i.e. RNAi-mediating, shRNA molecules. Naturally occurring transcriptional stop elements are well-known in the art such as, for example, poly-adenylation signals in eukaryotes.

“Recombinase recognition sequences” as used in the present invention relate to sequences which are recognized by enzymes capable of mediating site-specific recombination. Recombination involves enzyme-mediated cleavage of a DNA double strand and subsequent ligation of the cleaved DNA double strand to either the same or another equally cleaved DNA strand. The recombinase recognition sequence has a sequence motif which is specifically recognized by a recombinase and further contains a sequence motif which matches exactly with the terminal end of a DNA molecule for subsequent ligation, wherein recognition and matching motif can be overlapping or be the same. Thus, recombination can be used to excise or introduce a DNA segment by cleavage and ligation of dsDNA. Preferred are recombination recognition sequences which are aligned such as to allow for excision of the transcriptional stop elements, i.e., for example, using IoxP sequences as direct repeats instead of inverted repeats. The above applies mutatis mutandis to other embodiments herein.

The combination of DNA segments of the present invention relates to DNA segments which are suitable for efficient knockdown of target genes upon the excision of the transcriptional stop elements by the activity of a recombinase as described herein resulting in the transcription of shRNA molecules. Thus, said combination relates to DNA segments comprising DNA sequences which are accessible for the cellular transcriptional machinery leading to expression of shRNA molecules within a eukaryotic cell. The transcription process is preferably mediated by RNA polymerase III. The structure and function of short hairpin RNA molecules are well-known to the skilled person and methods for production and design are described, for example, in Paddison et al., (2002), Genes Dev., 16(8), 948-958; McIntyre et Fanning, (2006), BMC Biotechnol., 6:1.

Short hairpin RNA is capable of mediating gene knockdown just like any dsRNA in a process termed RNA interference (Fire et al., (1998), Nature, 391, 806-811). Once an shRNA molecule is transcribed in the cell, it is cleaved by an RNase III—like enzyme (Dicer), into double stranded small interfering RNAs (siRNA) loosing its loop structure. In an ATP dependent step, the siRNAs become integrated into a multi-subunit protein complex, commonly known as the RNAi induced silencing complex (RISC), which guides the siRNAs to the target RNA sequence (Nykanen et al., (2001), Cell, 107, 309-321). At some point during the integration phase the siRNA duplex unwinds, and the antisense strand remains bound to RISC and directs degradation of the complementary mRNA sequence by a combination of endo- and exonucleases (Martinez et al., (2002), Cell, 110, 563-574).

Efficiency of the knockdown may be measured by methods well-known in the art, for example, by measuring the protein or mRNA levels of the target genes before and after knockdown or by reporter gene assays. Preferably, the efficiency of the knockdown of the target genes mediated by the DNA molecule of the present invention is similar or preferably equal for either of said genes and knocks down at least 10%, at least 20%, at least 30%, at least 40%, at least 50%, at least 60%, at least 70%, at least 80% and preferably at least 90% or 95% of the target gene product relative to a control. Most preferred a complete knockdown of the target genes is envisaged.

Although RNAi-mediated gene knockdown has been the focus of research in a variety of fields, the present investigators surprisingly show regulated, conditional and efficient expression of two different shRNA molecules transcribed from one DNA molecule leading to simultaneous knockdown of two genes. The novel technique allows for conditional expression of two or more shRNA molecules upon recombinase-mediated activation.

Exemplarily, the present inventors impaired expression of two different genes by coupling two short hairpin RNAs (shRNA), each specific for one gene, in a row in one expression vector. To achieve a conditional activation of the shRNAs they used the Cre/IoxP site specific recombination system. The invention comprises additionally to the wild type IoxP sites, a mutated Iox site, called Iox2272 (Araki et al., Nucleic Acids Res., 30 (19), e103 (2002)), which cannot recombine with the wild type IoxP site. One of the used shRNAs is interrupted by a IoxP flanked stop cassette and the second shRNA is interrupted by a Iox2272 flanked stop cassette. Contact with Cre recombinase leads to recombination of the IoxP sites in one shRNA and of the Iox2272 in the other shRNA. In this recombination step the stop cassettes are cut out and RNAi is activated engaging the two targeted genes with the now active shRNAs. The activity pattern of Cre recombinase allows a control of the targeted tissues and cell types. By placing the conditional RNAi constructs into the defined genetic Rosa26 locus and by using recombinase mediated cassette exchange (RMCE) they were able to produce transgenic animals displaying a high rate of knockdown of the two targeted genes.

In conclusion, the present invention provides a technique for easy conditional silencing of two or multiple genes or gene families. This is important in a variety of fields, especially with regard to transgenic animals used as disease models or to generally assess gene function. Thus, an obvious application for the present invention is the production of shRNA vector mice using the large collection of mouse strains already carrying an expressible recombinase gene under the control of a tissue-specific promoter, for example, the Cre recombinase, which will allow for fast generation of a variety of different knockdown mouse strains as compared to the laborious, time-consuming method of generating traditional knockout mouse strains, which moreover only seldom are double knockouts. Furthermore, double knockouts cannot be produced within one step. Due to the compatibility of the combination of DNA segments with RMCE, i. e. recombination-mediated cassette exchange as described in detail further below herein, being a single-copy approach using defined and well-characterized genetic loci is available, which has the advantage of reliably generating double knockdown mice showing a well-definable, ubiquitous or tissue-specific knockdown phenotype as compared to knockout mice generated through homologous recombination.

In a preferred embodiment of the combination of DNA segments of the invention, the DNA segments are contained in the same DNA molecule.

In accordance with the invention said combination of DNA segments is preferably contained in the same DNA molecule, said segments being placed next to each other in the same or opposite direction. Said DNA molecule may contain further sequence elements flanking said combination of DNA segments of the invention. Such sequence elements can be, for example and without limitation, elements allowing for integration into another DNA molecule, such as, restriction sites or recombinase recognition sites.

In a preferred embodiment of the combination of DNA segments of the invention, said combination of DNA segments is part of a vector.

In accordance with the present invention, the DNA segments can be part of the same vector or can each be part of different vectors, wherein in the latter case both vectors have to be present at the same time to allow for simultaneous knockdown of two target genes. Preferably, each of the DNA segments is part of the same vector and arranged as described above when being contained in the same DNA molecule.

Preferably, the a vector is a plasmid, cosmid, virus, bacteriophage or another vector used, e.g., conventionally in genetic engineering.

The DNA segments may further be inserted into several commercially available vectors to be part thereof. Non-limiting examples include prokaryotic plasmid vectors, such as the pUC-series, pBluescript (Stratagene), the pET-series of expression vectors (Novagen) or pCRTOPO (Invitrogen) and vectors compatible with an expression in mammalian cells like pREP (Invitrogen), pcDNA3 (Invitrogen), pCEP4 (Invitrogen), pMC1neo (Stratagene), pXT1 (Stratagene), pSG5 (Stratagene), EBO-pSV2neo, pBPV-1, pdBPVMMTneo, pRSVgpt, pRSVneo, pSV2-dhfr, plZD35, pLXIN, pSIR (Clontech), pIRES-EGFP (Clontech), pEAK-10 (Edge Biosystems) pTriEx-Hygro (Novagen), pbsU6 and pClNeo (Promega).

The DNA segments may also be inserted into vectors to be part thereof such that a translational fusion with another DNA molecule is generated. The other DNA molecule may encode another protein which may e.g. mediate the recombination event leading to the excision of the transcriptional stop elements.

For vector modification techniques, see Sambrook, Molecular Cloning, A Laboratory Manual, Cold Spring Harbor Laboratory (1989) N.Y. Generally, vectors can contain one or more origin of replication (ori) and inheritance systems for cloning or expression, one or more markers for selection in the host, e. g., antibiotic resistance, and one or more expression cassettes. Suitable origins of replication (ori) include, for example, the Col E1, the SV40 viral and the M 13 origins of replication.

The coding sequences inserted besides the DNA segments in the vector can e.g. be synthesized by standard methods, or isolated from natural sources. Ligation of the coding sequences to transcriptional regulatory elements and/or to other amino acid encoding sequences can be carried out using established methods. Transcriptional regulatory elements (parts of an expression cassette) ensuring expression eukaryotic cells are well known to those skilled in the art. These elements comprise regulatory sequences ensuring the initiation of the transcription (e. g., translation initiation codon, promoters, enhancers, and/or insulators), internal ribosomal entry sites (IRES) (Owens, Proc. Natl. Acad. Sci. USA 98 (2001), 1471-1476) and optionally poly-A signals ensuring termination of transcription and stabilization of the transcript. Additional regulatory elements may include transcriptional as well as translational enhancers, and/or naturally-associated or heterologous promoter regions. Preferably, said coding sequences are operatively linked to such expression control sequences allowing expression in prokaryotes or eukaryotic cells. The vector may further comprise nucleotide sequences encoding secretion signals as further regulatory elements. Such sequences are well known to the person skilled in the art. Furthermore, depending on the expression system used, leader sequences capable of directing the expressed polypeptide to a cellular compartment may be added to the coding sequence of the polynucleotide of the invention. Such leader sequences are well known in the art.

Possible examples for regulatory elements ensuring the initiation of transcription comprise the cytomegalovirus (CMV) promoter, SV40-promoter, RSV-promoter (Rous sarcome virus), the lacZ promoter, the gall° promoter, human elongation factor 1α-promoter, CMV enhancer, CaM-kinase promoter, the Autographa californica multiple nuclear polyhedrosis virus (AcMNPV) polyhedral promoter, the SV40-enhancer or a U6 or H1 gene promoter. Examples for further regulatory elements of prokaryotes and eukaryotic cells comprise transcription termination signals, such as SV40-poly-A site or the tk-poly-A site or the SV40, lacZ and AcMNPV polyhedral polyadenylation signals, downstream of the polynucleotide.

Furthermore, it is preferred that the vector comprises a selectable marker. Examples of selectable markers include neomycin, ampicillin, and hygromycine, kanamycine resistance and the like. Specifically-designed vectors allow the shuttling of DNA between different hosts, such as bacteria-fungal cells or bacteria-animal cells (e. g. the Gateway® system available from Invitrogen).

An expression vector in accordance with this invention is capable of directing the replication, and the expression of the DNA segments of this invention encoding shRNA molecules. Suitable expression vectors which comprise the described regulatory elements are known in the art such as Okayama-Berg cDNA expression vector pcDV1 (Pharmacia), pRc/CMV, pcDNA1, pcDNA3 (In-Vitrogene), pSPORT1 (GIBCO BRL), or pGEMHE (Promega), or prokaryotic expression vectors, such as lambda gt11, pJOE, the pBBR1-MCS-series, pJB861, pBSMuL, pBC2, pUCPKS, pTACT1, pET vector (Novagen) or, preferably, expression vectors containing RNA Polymerase III driven promoters like pSUPER (Brummelkamp, et al., Science, 296, 550-553 (2002), pShag (Paddison, et al., Nat Methods, 1, 163-167 (2004) or the pBS-U6 vector (as used in the appended example).

The combination of DNA segments of the invention being part of the vectors as described herein above may be designed for direct introduction or for introduction via liposomes, phage vectors or viral vectors (e.g. adenoviral, retroviral) into the eukaryotic cell. Additionally, baculoviral systems or systems based on Vaccinia Virus or Semliki Forest Virus can be used as eukaryotic expression system for the combination of the DNA segments of the invention.

A typical mammalian expression vector may—besides the combination of DNA segments of the invention—further contain a promoter element, which mediates the initiation of transcription of mRNA, a protein coding sequence, and signals required for the termination of transcription and polyadenylation of the transcript. Moreover, elements such as origin of replication, drug resistance gene, regulators (as part of an inducible promoter) may also be included. Additional elements might include enhancers, Kozak sequences and intervening sequences flanked by donor and acceptor sites for RNA splicing. Highly efficient transcription can be achieved with the early and late promoters from SV40, the long terminal repeats (LTRs) from retroviruses, e.g., RSV, HTLVI, HIVI, and the early promoter of the cytomegalovirus (CMV). However, cellular elements can also be used (e.g., the human actin promoter). Suitable expression vectors for use in practicing the present invention include, for example, vectors such as pSVL and pMSG (Pharmacia, Uppsala, Sweden), pRSVcat (ATCC 37152), pSV2dhfr (ATCC 37146) and pBC12MI (ATCC 67109), IpSUPER (Brummelkamp, et al., Science, 296, 550-553 (2002)), pShag (Paddison, et al., Nat Methods, 1, 163-167 (2004)) or the pBS-U6 vector (as used in the appended example; SEQ ID NO: 8). Mammalian host cells that could be used include, human Hela, 293, H9 and Jurkat cells, mouse NIH3T3 and C127 cells, Cos 1, Cos 7 and CV1, quail QC1-3 cells, mouse L cells and Chinese hamster ovary (CHO) cells. Alternatively, the shRNA molecules can be expressed in stable cell lines that contain the combination of DNA segments integrated into a chromosome. The co-transfection with a selectable marker such as dhfr, gpt, neomycin, hygromycin allows the identification and isolation of the transfected cells. The transfected nucleic acid can also be amplified to express large amounts of the encoded shRNAs. The DHFR (dihydrofolate reductase) marker is useful to develop cell lines that carry several hundred or even several thousand copies of the vectors carrying the combination of the DNA segments of the invention. Another useful selection marker is the enzyme glutamine synthase (GS) (Murphy et al. 1991, Biochem J. 227:277-279; Bebbington et al. 1992, Bio/Technology 10:169-175). Using these markers, the mammalian cells are grown in selective medium and the cells with the highest resistance are selected. As indicated above, the expression vectors will preferably include at least one selectable marker. Such markers include dihydrofolate reductase, G418 or neomycin resistance for eukaryotic cell culture and tetracycline, kanamycin or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells; insect cells such as Drosophila S2 and Spodoptera Sf9 cells; animal cells such as CHO, COS, 293 and Bowes melanoma cells; and plant cells. Appropriate culture mediums and conditions for the above-described host cells are known in the art.

In another preferred embodiment of the combination of DNA segments of the invention, the recombinase recognition sequences are Iox sequences encompassing a wild type IoxP sequence and a mutant IoxP sequence.

The term “Iox sequences” as used in accordance with the present invention relates to sequence motifs which are specifically recognized by the Cre recombinase, a type I topoisomerase from the P1 bacteriophage. The “Iox sequences” referred to herein encompass the wild type IoxP recognition sequence consisting of 34 by wherein two 13 by palindromes (inverted repeats) are flanking an 8 by core region and wherein the wild type IoxP recombinase recognition has the sequence of SEQ ID NO.: 2. Further encompassed are mutant IoxP sequences, wherein the mutant IoxP sequences include sequences with not more than 8 nucleotide substitutions relative to the wild type IoxP sequence of SEQ ID NO.: 2. Mutant IoxP sequences with 1, 2, 3,4,5,6 or 7 nucleotide substitutions are deliberately envisaged. In accordance with the invention, the wild type IoxP and the mutant IoxP sequence are not compatible for recombination with each other, but are exclusively recombinable by the activity of the Cre recombinase with Iox sequences with an identical sequence. Several Cre recombinases are presently known and derived via mutagenesis from the wild type bacteriophage P1 Cre recombinase in order to change features like, for example, nuclear localization or translation efficiency in mammalian cells. The skilled person is in the position to identify suitable combinations of Iox sequences to be recognized by a single Cre recombinase which can be used in accordance with the present invention.

In a more preferred embodiment of the combination of DNA segments of the present invention, the recombinase is a Cre recombinase having the sequence of SEQ ID NO.: 1.

Said Cre recombinase is the wild type Cre recombinase as originally isolated from bacteriophage P1 (SEQ ID NO.: 1) and belongs to the family of DNA recombinases which catalyze site-specific recombination between two identical Iox sequences.

In another more preferred embodiment of the combination of DNA segments of the invention, either the first or the second type of Iox sequences has the sequence of SEQ ID NO.: 2.

Said Iox sequence is the wild type IoxP sequence (SEQ ID NO.: 2). It is recombined only at reduced efficiency with many mutant IoxP sequences but does not recombine (below 5%, preferrably below 1% efficiency as compared to IoxP×IoxP recombination as assayed in vitro (Lee and Saito, Gene, 216, 55-65 (1998)) with selected mutant IoxP sequences in the presence of a Cre recombinase. The recombination occurs at optimal efficiency with a second wild type IoxP site having the identical sequence, i.e. SEQ ID NO.: 2, in accordance with the invention.

Selected mutant Iox sites in accordance with the present invention are mutant Iox sites which recombine with each other in the presence of Cre recombinase with an efficiency of at least 10% relative to the efficiency of the wild type IoxP×IoxP recombination reaction. Advantageously, said recombination efficiency is at least 20%, 30%, 40%, 50%, 60%, 70%, 80%, 90% and most preferably at least 100%. For example, said mutant Iox sequences can be Iox 5171 (SEQ ID NO: 18), Iox 5271 (SEQ ID NO: 19), Iox 5371 (SEQ ID NO: 20), Iox 5172 (SEQ ID NO: 21), Iox 5272 (SEQ ID NO: 22), or Iox 5372 (SEQ ID NO: 23) as published in Lee and Saito, Gene, 216, 55-65 (1998). Further examples include m2 (SEQ ID NO: 24), m3 (SEQ ID NO: 25), m7 (SEQ ID NO: 26), m11 (SEQ ID NO: 27) as published in Langer et al., Nucleic Acids Res. 30, 3067-77 (2002).

In another more preferred embodiment of the combination of DNA segments of the invention, either the first or the second type of Iox sequences has the sequence of SEQ ID NO.: 3.

Said Iox sequence is a mutant IoxP sequence (Iox2272; Araki et al., Nucleic Acids Res., 30 (19), e103 (2002)) and does not recombine with the wild type IoxP sequence having the sequence of SEQ ID: NO.: 2 in the presence of Cre recombinase. The recombination only occurs with a second mutant IoxP site having the identical sequence, i.e. SEQ ID NO.: 3, at an efficiency of at least 10% as compared to the recombination of wild type IoxP sequences, in accordance with the invention.

In a preferred embodiment of the combination of DNA segments of the invention, the transcriptional stop element contains sequences that interfere with RNA polymerase III driven transcription.

RNA polymerase III is a polymerase which transcribes DNA to synthesize ribososmal 5S rRNA, tRNA and other small RNAs.

Transcriptional stop elements of RNA polymerase have been first reported by Brown et Brown, (1976), J. Mal. Biol., 102, 1-14, and the signal has been characterized as four or more consecutive thymidine residues on the non-coding strand and G+C richness of the flanking DNA of the 5 S rRNA gene of frog cells. A transcriptional stop element that interferes with RNA polymerase III transcriptional activity in accordance with the invention are, for example, short repeats of at least 5 or more thymidine bases. Preferred are repeats of at least 6 thymidine bases.

In another preferred embodiment of the combination of DNA segments of the present invention, the promoters are promoters of genes transcribed by RNA polymerase III.

Genes being transcribed by RNA polymerase III are preferably so-called “house-keeping” genes the expression of which is required in all cell types and most developmental and environmental conditions. Thus, the regulation of RNA polymerase III is primarily tied to cell growth and the cell cycle and less complex than the initiation and regulation of the transcriptional process mediated by RNA polymerase II.

It is envisaged that a promoter which mediates the activity of RNA polymerase Ill and thus is cell-type independent and requires less co-factors is especially suitable for the expression of shRNA molecules in accordance with the invention. Such promoters are, for example, tRNA gene regulating promoters, rRNA, snRNA and miRNA gene regulating promoters.

Accordingly, in a more preferred embodiment of the combination of DNA segments of the invention, the promoters are selected from the group consisting of U6 or H1 gene promoters (Park and Kunkel, Biochem Biophys Res Commun, 214, 934-940 (1995); Myslinski, et al., Nucleic Acids Res, 29, 2502-2509 (2001)). These promoters are driven by RNA Polymerase III and allow the efficient production of short RNA molecules with defined ends and no sequence additions, since the termination signal of RNA Polymerase III consists of a stretch of thymidine residues. Both promoters are short (100-200 bp) and contain two sequence elements essential for transcription, the TATA box and the proximal sequence element (PSE).

In a more preferred embodiment of the combination of DNA segments of the invention, said DNA molecule containing said combination of DNA segments comprises further elements allowing for stable integration of said molecule into the genome of a non-human animal.

“Stable integration” in accordance with the present invention relates to the incorporation of a DNA sequence into the genome of a non-human mammal. This is in contrast to DNA sequences which are introduced into a cell and are only transiently transcribed and expressed extrachromosomally.

Analysis of function and expression of transfected genes may require the stable integration of the transfected DNA into the host genome. A variety of methods exists to transfect cells and said methods are well-known to the skilled person (see, for example, Sambrook, Molecular Cloning, A Laboratory Manual, Cold Spring Harbor Laboratory (1989) N.Y. and Ausubel, Current Protocols in Molecular Biology, Green Publishing Associates and Wiley Interscience, N.Y. (1889)). In the present case, it is preferred that the combination of DNA segments of the invention is stably integrated into the genome of a non-human mammal or into the genome of any eukaryotic cell in vitro or in vivo. The stable integration allows for the continuous and persistent expression of shRNA molecules with subsequent RNAi-mediated knockdown of the target genes and may be achieved by a variety of methods including, for example, pronuclear injection, lentiviral infection or electroporation with for example, subsequent homologous recombination-mediated integration. In this case, elements for stable integration generally consist of sequences complementary to sequences of the targeted genome. For example, a shRNA vector could be integrated into the ubiquitous active Rosa26 locus by flanking the vector with Rosa26 homology sequences and subsequent introduction into embryonic stem cells.

In a more preferred embodiment of the combination of DNA segments of the invention, said further elements allow for site-specific integration.

“Site-specific integration” as used herein relates to the stable integration of a combination of DNA segments of the invention at a specific defined genetic locus of the genome of a non-human mammal or any eukaryotic cell in vitro or in vivo.

Preferably, the combination of DNA segments of the present invention is integrated site-specifically. Site-specific incorporation allows for controlling the function and/or activity of the integrated combination of DNA segments. The transcriptional activity of the genetic locus chosen for integration can directly influence the expression of the integrated combination of DNA segments and further the copy number of the combination of DNA segments in the genome can be limited according to the occurrence of said chosen genetic locus. The person skilled in the art is in the position to choose an appropriate genetic locus for integration of the combination of DNA segments of the invention according to his preferences regarding function and/or activity of said combination of DNA segments.

A preferred approach to integrate the combination of DNA segments of the invention stably and site-specifically is the RMCE (recombinase-mediated cassette exchange) approach. In this approach, the donor, i.e. the combination of DNA segments of the invention, and the target sequences, i.e. cassette at the genetic locus, or commonly referred to as cassettes, are each flanked by recognition sites for site-specific recombinases. Crossover events occurring on both sides of the donor and target sequences result in a clean exchange of the target cassette with the donor cassette. To ensure stability of the integrant, cassettes can be flanked by either inverted or preferably heterotypic recognition sequences, with both strategies allowing exchange between the donor and target but preventing excision of either cassette. Further this strategy ensures the integration of the donor cassette itself, rather than the backbone. Because RMCE involves the exchange of sequences rather than the simple insertion of a transgene, it provides two advantages: (a) only the sequence of the DNA sequences within the cassette, i.e. the combination of DNA segments of the invention, is integrated, in contrast to strategies that use a single recombinase recognition site leading to incorporation of the entire donor sequence; and (b) integration of sequences that do not on their own produce a phenotype is possible, thus a successful exchange can be detected simply by the loss of a marker carried by the target cassette and/or the gain of a marker carried by the donor cassette. Both of these advantages reduce the amount of extraneous sequence required for integration, thereby simplifying cloning steps and precluding any requirement for nearby transcriptional units that may influence gene expression.

In another more preferred embodiment of the combination of DNA segments of the invention, the further elements for stable and site-specific integration into the genome are sequences suitable for integration of the combination of DNA segments through recombination.

“Recombination” as used herein has been described supra and is the method preferably used for integration of the combination of DNA segments into the targeted genome. More preferred are sequences which allow for RMCE leading to the integration of the combination of DNA segments of the invention. These sequences may, for example, be encompassed in a donor cassette used for RMCE wherein said cassette comprises a pair of recombinase recognition sites flanking both a resistance gene and the combination of DNA segments of the invention.

In accordance with the above, in another more preferred embodiment of the combination of DNA segments of the invention, the site of integration is a genetic locus comprising sequences suitable for integration of the combination of DNA segments through recombination.

To allow for stable and site-specific integration of the combination of DNA segments of the invention into a genome through recombination the genetic locus comprises sequences which are identical to the sequences suitable for integration through recombination of the combination of DNA segments of the invention. Thus, the combination of DNA segments may be integrated into the genome via a recombination event. Preferred are sequences which allow for RMCE leading to the incorporation of the combination of DNA segments of the invention. These sequences may, for example, be encompassed in an acceptor cassette used for RMCE wherein said cassette comprises a pair of recombinase recognition sites flanking a resistance gene under the control of a promoter regulating the expression of said resistance gene.

In a most preferred embodiment of the combination of DNA segments of the invention, said sequences suitable for integration through recombination are recognition sites for enzymes mediating recombination events.

Said “recognition sites for enzymes mediating recombination events” have been described supra (cf. “Recombinase recognition sequences”). The recognition sites for the stable and site-specific integration of the combination of DNA segments are recognized by a different enzyme than the recombinase recognition sequences flanking the transcriptional stop element. In other words, an enzyme used for excising the transcriptional stop elements may not be able to mediate stable and site-specific integration of the combination of DNA segments of the invention. Preferably said enzyme is an integrase, such as phiC31 Integrase.

Accordingly, in a more preferred embodiment of the above embodiment of the combination of DNA segments of the invention, the enzymes mediating recombination events are DNA recombinases or integrases.

The mode of action of DNA recombinases and integrases has been described supra. Recombinases used in accordance with the present invention are, for example, FLP recombinase, that mediates recombination between identical FRT recognition sites. Integrases as used in accordance with the present invention are, for example, phage integrases that mediate unidirectional site-specific recombination between two DNA recognition sequences, the phage attachment site (attP), and the bacterial attachment site (attB). Some phage integrases require host cofactors for strand cleavage and ligation. The recognition sites are relatively short, yet long enough to be specific on a genomic scale and are thus usable in the context of large eukaryotic genomes. Preferably, the phiC31 integrase is used to mediate stable and site-specific integration of the combination of DNA segments of the invention, wherein the genetic locus comprises attP sequences, the combination of DNA segments of the invention comprises attB sequences and by the activity of phiC31 integrase are recombined.

In another more preferred embodiment of the combination of DNA segments of the invention, said DNA molecule containing said combination of DNA segments is integrated into a genetic locus having a mild or ubiquitous transcriptional activity.

A genetic locus according to the invention encompasses any point of the genome, preferably displaying transcriptional activity. As described herein, promoters can regulate the expression of genes due to their internal structure which mediates attachment and dissociation of inter alia transcription factors. Hence, the presence and structure of a promoter determines the degree of transcriptional activity of a genetic locus. The rate of expression of shRNA molecules will thus be at least partially dependent on the transcriptional activity of the genetic locus for integration. Thus, the degree of knockdown of the targeted genes can be controlled. For example, integration at a genetic locus having a ubiquitously transcriptional activity may lead to a high degree of knockdown of the targeted genes whereas a genetic locus with less transcriptional activity may lead to a lower degree of knockdown of said genes. The person skilled in the art will be in the position to choose using his general common knowledge a suitable genetic locus displaying the desired level of transcriptional activity. Further he may retrieve information on said genetic loci using well-known databases such as maintained by the National Center for Biotechnology Information (NCBI; http://www.ncbi.nlm.nih.gov/) or the EMBL-EBI and the Wellcome Trust Sanger Institute (Ensembl; http://www.ensembl.org/index.html).

In a more preferred embodiment of the combination of DNA segments of the invention, said combination of DNA segments is integrated at the Rosa26 locus or Hypoxanthin-Phosphoribosyl-Transferase (HPRT) locus. These loci are widely or ubiquitously expressed in mouse tissues and also enable the undisturbed expression of transgenic vectors inserted into these loci.

In a further embodiment the invention relates to a method of producing a transgenic non-human animal, the method comprising the steps of (a) integrating the DNA molecule containing said combination of DNA segments of the invention into the genome of a non-human animal; and of (b) crossing said animal with an animal transgenic for an expressible recombinase gene, wherein said recombinase recognizes the recombinase recognition sequences flanking the transcriptional stop element of said DNA segments.

The term “transgenic non-human animal” as used in accordance with the invention relates to an animal in which there has been a deliberate modification of its genome.

A method for the production of a transgenic non-human animal, for example, a transgenic mouse, comprises introduction of the combination of DNA segments of the invention into a germ cell, an embryonic cell, stem cell or an egg or a cell derived therefrom. Production of transgenic embryos and screening of those can be performed, e.g., as described by A. L. Joyner Ed., Gene Targeting, A Practical Approach (1993), Oxford University Press. The DNA of the embryonic membranes of embryos can be analyzed using, e.g., Southern blots with an appropriate probe.

Integration of the combination of DNA segments into the genomes of non-human animals can be achieved by a variety of methods well-known to the person skilled in the art. General methods for making transgenic non-human animals are described in the art, see, for example, WO 94/24274. Such methods include, but are not limited to (a) DNA microinjection, (b) embryonic stem cell-mediated gene transfer and (c) retrovirus-mediated gene transfer.

(a) DNA microinjection involves the direct microinjection of a chosen DNA construct from any species into the pronucleus of a fertilized ovum. However, the insertion of the DNA is a random process and there is a high probability that the introduced combination of DNA segments does not insert itself into site in the host genome that will permit its expression. The manipulated ovum is transferred into the oviduct of a recipient female or artificially induced recipient female.

(b) Embryonic stem cell-mediated gene transfer involves prior insertion of the combination of DNA segments by homologous recombination into an in vitro culture of embryonic stem (ES) cells. These cells are then incorporated into an embryo at the blastocyst stage of development. The result is a chimeric animal. ES cell-mediated gene transfer is the preferred method for gene inactivation. It has the advantage of allowing precise targeting of defined mutations in the gene via homologous recombination.

(c) This gene transfer is mediated by means of a carrier or vector, generally a virus or a plasmid to increase the probability of expression. Retroviruses are commonly used as vectors to transfer genetic material into the cell, taking advantage of their ability to infect host cells in this way. Offspring derived from this method are chimeric as not all cells carry the retrovirus. Transmission of the transgene is possible only if the retrovirus integrates into some of the germ cells.

Any of the above methods includes testing the first generation (F1) of animals for the expression of the shRNA molecule directly or indirectly. Chimeras may then need to be inbred for a several more generations to obtain homozygous animals.

Preferred for making transgenic non-human animals of the invention (which include homologously targeted non-human animals) are embryonic stem cells (ES cells). Murine ES cells, such as, for example, AB-1 line grown on mitotically inactive SNL76/7 cell feeder layers (McMahon and Bradley, Cell 62:1073-1085 (1990)) essentially as described (Robertson, E. J. (1987) in Teratocarcinomas and Embryonic Stem Cells: A Practical Approach. E. J. Robertson, ed. (Oxford: IRL Press), p. 71-112) or embryonic fibroblasts may be used for homologous gene targeting. Other suitable ES lines include, but are not limited to, the E14 line (Hooper et al., Nature 326:292-295 (1987)), the D3 line (Doetschman et al., J. Embryol. Exp. Morph. 87:27-45 (1985)), the CCE line (Robertson et al., Nature 323:445-448 (1986)), the AK-7 line (Zhuang et al., Cell 77:875-884 (1994)), or the IDG26.10-3 ES cells (described in the appended example).

The success of generating a mouse line from ES cells bearing the combination of DNA segments of the invention depends on the pluripotence of the ES cells (i. e., their ability, once injected into a host developing embryo, such as a blastocyst or morula, to participate in embryogenesis and contribute to the germ cells of the resulting animal). The blastocysts containing the injected ES cells are allowed to develop in the uteri of pseudopregnant non-human females and are born, e.g. as chimeric mice. The resultant transgenic mice are chimeric for cells having said combination of DNA segments or not and are backcrossed and screened for the presence of the correctly targeted combination of DNA segments by PCR or Southern blot analysis, for example, on tail biopsy DNA of offspring so as to identify transgenic mice homozygous for the combination of DNA segments.

Preferably and as outlined in the appended example, the vector comprising the combination of DNA segments of the invention as part of the donor cassette for RMCE and a recombinase expression vector are co-electroporated into ES cells carrying the acceptor cassette for RMCE. After transfection, the ES cells are selected for their ability to survive treatment with an agent for selection. ES cells having undergone successful RCME express the resistance gene contained within the donor cassette, wherein the agent for selection is chosen according to the selected resistance gene, e.g., neomycin positive ES cells will survive treatment with G418. Subsequently, single colonies are picked and maintained undifferentiated. Finally, ES cells are introduced as described above into blastocysts and mice with germline transmission are crossed homozygous for the recombinase recognition sequence hairpin allele. These homozygous mice are then crossed to a recombinase-expressing mouse strain to mediate the activation of the hairpins in the desired tissue(s), wherein said recombinase recognizes the recombinase recognition sequences flanking the transcriptional stop elements of the combination of DNA segments of the invention. Preferably, said recombinase is under the control of a tissue-specific or ubiquitous promoter.

The transgenic non-human animals may, for example, be transgenic mice, rats, hamsters, dogs, monkeys, rabbits, pigs, or cows.

In another embodiment the invention relates to a genetically engineered non-human animal transgenic for the DNA molecule containing said combination of DNA segments of the invention and an expressible recombinase gene.

Said animal is preferably produced by the method of the invention and can be used in accordance with the invention, for example but not limited to, in a method of simultaneously knocking down two genes or for identification of a combination of target genes as a potential drug target as described herein below.

In a preferred embodiment of the genetically engineered non-human animal of the invention, the recombinase is expressed under the control of a tissue-specific promoter.

The term “tissue-specific promoter” as used in accordance with the present invention relates to promoters, which are genomic DNA sequences that enable and control transcription of the gene(s) they are associated with. The concerted regulation of gene expression is especially important in multicellular organisms. Thus, promoters are involved in a complex coordination of transcription under all conceivable spatio-temporal-conditional circumstances. This is achieved by their internal structure, consisting of arrays of individual protein (e.g., transcription factors) binding sites, that form a hierarchical structure of modules, i.e. functionally important and transferable combinations. Regarding tissue-specificity it is well-known in the art that gene expression varies at different stages in the development of cells (e.g. senescence, differentiation) and varies from cell type to cell type. Cell types have—due to differential expression—characteristic profiles of the genes expressed. The characteristic array of expressed genes can be used for classifying a cell, i.e. what kind of cell, what developmental stage, normal or diseased. A tissue-specific promoter according to the invention is a promoter which can be used to classify cells, i.e. differentiate cells, and is preferably exclusively active in a single class of cells relative to other cells or cell classes. The person skilled in the art is aware of several tissue-specific promoters and further tissue-specific promoters can be found according to methods as described, for example, in Chen et al., (2006), Nucleic Acids Research, 34, database issue D104-D107. Further, genetically engineered non-human animals transgenic for a recombinase under the control of a tissue-specific promoter can be obtained from several well-known facilities such as, for example, The Jackson Laboratory (Bar Harbor, Me., USA), B & K Universal Limited (Hull, England), Charles River Laboratories, Inc. (Wilmington, Mass., USA), Harlan (Indianapolis, Ind., USA) or Taconic Farms, Inc. (New York, N.Y., USA).

In a preferred embodiment of the method or the genetically engineered non-human animal of the invention, the non-human animal is a rodent.

Rodents, for example, rats and mice, have been shown to be suitable as research tool (e.g. disease model) for several reasons. For example, mice adapt well to laboratory housing and can be housed socially or individually. Significant numbers can be housed in relatively little space because of their small body size. Further, they possess a surprising genetic similarity to humans—approximately 85% similarity on a by to by basis. Furthermore, it has been shown that 90% of genes linked to diseases are the same in mice as in humans. These features—the similar gene sequence, genetic content and arrangement of genes—combined with a rapid rate of reproduction, make rodents, preferably mice, the non-human animal of choice for genetic manipulation as envisaged and described herein.

Accordingly, in a more preferred embodiment of the method or the genetically engineered non-human animal of the invention, the rodent is a mouse.

Further, in an embodiment the present invention relates to a eukaryotic cell genetically engineered with the combination of DNA segments of the invention.

The cell can be any eukaryotic cell wherein the combination of DNA segments has been preferably introduced into the genome of said cell via recombination or is maintained extrachromosomally as part of vectors and upon excision of the transcriptional stop elements through the activity of a recombinase as described herein above, shRNA molecules are expressed which knock down the expression of the target genes through RNAi. Also preferred is that the combination of DNA segments used for engineering said eukaryotic cell is contained in the same DNA molecule.

The term “eukaryotic” is meant to include yeast cells, cells of higher plants, insect cells and preferably mammalian cells, such as, for example, an ES cell as described herein. A combination of DNA segments of the invention can be used to transform or transfect said cell using any of the techniques commonly known to those of ordinary skill in the art and, for example, described in Sambrook, Molecular Cloning, A Laboratory Manual, Cold Spring Harbor Laboratory (1989) N.Y. and Ausubel, Current Protocols in Molecular Biology, Green Publishing Associates and Wiley Interscience, N.Y. (1889).

In another embodiment the invention relates to a method of simultaneously knocking down two genes in a eukaryotic cell comprising the steps of: (a) introducing the combination of DNA segments of the invention into a cell; (b) excising the transcription stop elements of (ab) and (bb) through the activity of a recombinase.

The combination of DNA segments of the invention may be introduced into a cell according to methods well-known to the person skilled in the art and described herein above and below. The mode of action of recombinases has also been described herein and is well-known in the art. Also preferred is that the combination of DNA segments used for knocking down two genes in said eukaryotic cell is contained in the same DNA molecule.

In a preferred embodiment of the cell of the invention or the above method of the invention, the recombinase is expressed in said cell.

Preferably, the functional recombinase is endogenously expressed in said cell or the gene encoding said recombinase has been introduced into the cell according to methods well-known in the art and maintained extrachromosomally, for example, on a plasmid or other vector, or stably integrated into the genome and can thus be expressed.

In a more preferred embodiment of the above cell or the method of invention, the recombinase is expressed under the control of a tissue-specific promoter.

As described herein above, tissue-specific promoters are confined in exerting their activity in a specific cell class. Thus, the use of a tissue-specific promoter as promoter for regulating the expression of the recombinase will lead to the expression of said shRNA molecules and subsequent knockdown of the target genes only in cells in which said promoter is active. This conditional knockdown is advantageous in a variety of therapeutic or experimental settings, for example, the study of tissue-specific diseases or the effect of a disease on a specific tissue or compartment of the body, generation of nonlethal mutations in non-human animal embryos, as outlined throughout the specification.

In a preferred embodiment of the method of the invention, the recombinase is exogenously introduced into the cell.

The recombinase intended to excise the transcription stop elements in order to allow for the expression of shRNA molecules may be added to the cell and, upon uptake, will exert activity within the cell. The person skilled in the art is aware of conditions generally suitable for uptake of proteins, including enzymes such as recombinases, into cells and methods to enhance said uptake as regards rate and amount wherein said enhancement may include artificially modifying proteins (see, for example, Patsch et Edenhofer, (2007), Handb Exp. Pharmacol., 178, 203-232). Furthermore, (s)he is also aware of cell lines naturally exhibiting the capacity of increased protein uptake relative to other cells. Such cells are, for example, cells like mucosal cells or intestinal cells. A number of mechanisms exist for the passage of proteins across the plasma membrane, including passive diffusion, facilitated diffusion, and active transport systems. Passive diffusion of proteins through the bilayer lipid structure of the plasma membrane is a function of the size, lipid solubility, and charge of the protein molecule. A further uptake mechanism is endocytosis. Endocytosis is a process whereby cells absorb material from the outside by engulfing it with their cell membrane. Endocytosis works with macromolecules or particulate matter beyond a certain size threshold.

In a further embodiment, the invention relates to a method of identifying a combination of two target genes as a potential drug target comprising the steps of: (a) determining different expression or activity of nucleic acid molecules or proteins in a cell exhibiting characteristics associated with a disease and in a normal cell; (b) knockdown of two genes according to the method of the invention in said cell exhibiting characteristics associated with a disease; and (c) determining the effect of the knockdown on said cell exhibiting characteristics associated with a disease; wherein a change in said disease characteristics is indicative that said combination of two target genes is a potential drug target.

The identification of drug targets aims at providing information on how to treat diseases. Diseases as used herein encompass without limitation any disease being triggered, exacerbated by and/or related in any way to the presence of endogenous factors (e.g. genetic predisposition) and/or ambient factors (e.g. bacteria, viruses). Said diseases may, for example, be associated with discomfort, dysfunction, distress, injury, disability, syndromes, infections, changes in behaviour, atypical variations in structure and function. While many diseases are biological processes with observable alterations of organ function or structure, others may primarily or exclusively involve alterations of behaviour.

Different expression or activity of nucleic acid molecules or proteins can be determined by methods well-known in the art (see, for example, “Molecular Cloning: A Laboratory Manual” by Sambrook et al. (Cold Spring Harbour Laboratory Press) or “Current Protocols in Molecular Biology” (Ausubel et al., Wiley and Sons, Inc); “Analysing Gene Expression, A Handbook of Methods: Possibilities and Pitfalls” by Stefan Lorkowski, Paul M. Cullen (eds.); Wiley-VCH, Weinheim. The methods preferably are highly sensitive and provide reproducible results. Examples of such methods are flow-cytometry, immunoassays (e.g., ELISAs), affinity mass spectrometry, preferably DNA- and protein-microarrays. Further, methods based on the detection of mRNA level alteration, which are in particular, methods based upon the polymerase chain reaction (real-time PCR) and related amplification technologies, such as NASBA and other isothermal amplification technologies, may be used.

As described above, characteristics of diseased cells can be any symptom known to be associated with a disease, as, for example, aberrant structure and/or function of the cell and/or as well as changes in gene expression commonly associated with a disease. Preferably the disease characteristics are detectable with any of the above methods regarding detection of different expression or activity of nucleic acid molecules or proteins. This is, for example, the case in cancer cells of various different cancers, which can be classified according to inter alia their gene expression profile. The person skilled in the art is well aware of further disease characteristics or may retrieve information from databases providing, for example, information on DNA- and/or protein arrays.

Determining the effect of a knockdown may be detection of any change of the disease characteristics, wherein disease characteristics can be a combination of several symptoms or only one symptom. Suitable methods for detecting the effect depend on the nature of the disease characteristic. For example, any of the above recited methods for determining the expression or activity of nucleic acid molecules or proteins can be used when a disease characteristic is the aberrant expression or activity of a nucleic acid molecule or protein relative to a control, as, for example, in cancer cells. Preferably, methods suitable to provide measurable and reproducible results may be used to detect the effect being a change of the disease characteristics. A change encompasses a decrease or increase of a factor which qualitatively and/or quantitatively describes said disease characteristics, and preferably involves the disappearance of said disease characteristics.

The above applies mutatis mutandis for the embodiments supra and infra.

In a preferred embodiment of the method of the invention, at least one of the genes is known to be associated with said disease.

Genes associated with a disease(s) are well-known to the person skilled in the art. Further, annotated records for known sequence variations in the human genome can be found at the Human Genome Variation database (HGVbase, http://hqvbase.cgb.ki.se/) and may provide further insight in genes associated with a disease. Said genes may be genes which are, for example, indicative of, causative for and/or contributory to a disease. The second gene can be any other gene, preferably a gene identified as being differently expressed or having a different activity according to step (a). Alternatively, said second gene may be located within the linkage disequilibrium (LD) block (as generated during haplotype analysis, e.g. for genomic screening for diseases) of the gene known to be associated with a disease, wherein the LD block is a DNA segment within which markers are in significant linkage disequilibrium with each other, which implies that there is low recombination activity within that block.

In another preferred embodiment of the method of the invention, the cell exhibiting characteristics associated with a disease is to be obtained from a patient.

Methods for obtaining samples containing diseased cells are well-known in the art and encompass, for example, isolation of blood cells, cells in the mucus, bile, stool, saliva and other easily accessible cells of the patient as well as cells obtained through surgery as part of tissue samples or by-products of surgery depending on the disease type. A cell obtained accordingly can be cultured according to methods well-known in the art as a primary cell line or modified, for example, immortalized to be used as a permanent cell line. Protocols for the isolation of different cell types, their culture conditions, and for the evaluation of the degree of differentiation of a primary cell culture are known in the art. In the case of tissue samples, mononuclear cells can be obtained, for example, by enzymatic digestion with enzymes such as collagenase, trypsin or pronase or any other enzyme breaking down the extracellular matrix. Another strategy involves placing the tissue sample in growth media, and cells that grow are available for tissue culture, the method being termed explant culture. A variety of methods and kits to isolate specific cell-types are well-known in the art and may be obtained, for example, from Qiagen or Miltenyibiotech.

Immortalizing a primary cell line may be achieved by random mutation or deliberate modification, such as, for example, artificial expression of the telomerase gene. Other methods are well-known to the person skilled in the art. There are numerous well established and characterized cell lines representative of particular cell types, both normal and diseased. Said cell lines may be obtained, for example, at the American Type Culture Collection (ATCC, www.atcc.org) or Deutsche Sammlung von Mikroorganismen and Zellkultur (DSMZ, www.dsmz.de).

Culture conditions vary from cell-type to cell-type and moreover, can result in different phenotypes being expressed for a particular cell-type. Generally, cells are grown and maintained at an appropriate temperature and gas mixture, i.e. typically 37° Celsius, 5% CO₂, in growth media (a) as irrigating, transporting and diluting fluid while maintaining intra- and extra-cellular osmotic balance, (b) that provides cells with water and certain bulk inorganic ions essential for normal cell metabolism, (c) which—combined with a carbohydrate, such as glucose—provides the principle energy source for cell metabolism and (d) which provides a buffering system to maintain the medium within physiologic pH range, i.e. cells are kept viable. The recipe of growth media varies greatly depending on cell-type and contains, for example and without limitation, growth factors, nutrient components, glucose, buffers to maintain pH and antifungizides and -biotics. Methods for culturing and maintaining cells in culture are well-known in the art and described, for example, in “Practical Cell Culture Techniques”, Boulton et Baker (eds), Humana Press (1992), ISBN 0896032140; “Human Cell Culture Protocols”, Gareth E. Jones, Humana Press (1996), ISBN 089603335X; growth media and other cell culture related material as well as instructions and methods for successful culturing of cells can, for example, be obtained at Sigma-Aldrich.

In a preferred embodiment of the above method of the invention, steps (a) to (c) are performed in a mouse.

As described herein above, mice have proven to be advantageous as a disease model for a variety of human diseases. Hence a variety of mouse lines and protocols have been developed to generate disease models in order to elucidate aspects of a target disease. Preferably, for determination of different expression or activity in step (a) a control mouse is used, i.e. a mouse without knockdown of two genes but manipulated to show symptoms of a disease. Said control mouse advantageously is a mouse only transgenic for an expressible recombinase gene. Further preferred, in step (b) the cell used is part of the transgenic mouse of the invention manipulated to show symptoms of a disease and determination of the effect in step (c) is performed in cells obtained from the transgenic mouse of the present invention relative to cells obtained form the control mouse of step (a). In other words, the transgenic mouse of the invention is used as a disease model and the effect of the knockdown of the two genes on the disease characteristics is compared to the disease characteristics of the control mouse also used as a disease model, wherein the disease models are the same. The skilled person will be able to obtain diseased cells of a such modified animals and determine different expression or activity of nucleic acid molecules or proteins according to any of the methods described herein and well-known to person skilled in the art.

In a preferred embodiment of the combination of DNA segments, the cell or the method of the invention, said eukaryotic cell is selected from the group consisting of non-human embryonic stem cells, cells contained in a tissue sample and a cell contained in a transgenic non-human mammal.

As described herein-above, embryonic stem cells are essential to produce transgenic animals of the invention. Further, the combination of DNA segments may be introduced into a eukaryotic cell being part of a tissue sample or of a transgenic non-human animal.

The present invention furthermore provides a pharmaceutical composition comprising the combination of DNA segments of the invention.

Finally, the present invention relates to the use of the combination of DNA segments of the invention for the preparation of a composition for gene therapy. Also provided is the combination of DNA segments of the invention for gene therapy.

The use of the combination of DNA segments of the invention encoding functional and expressible shRNA molecules is intended for the preparation of a composition for treating, preventing and/or delaying a disorder diagnosed by the method of the invention or known in the art. Gene therapy, which is based on introducing therapeutic DNA constructs into cells by ex vivo or in vivo techniques is one of the most important applications of gene transfer. Suitable vectors and methods for in vitro or in vivo gene therapy are described in the literature and are known to the person skilled in the art; see, e.g., Giordano, Nature Medicine 2 (1996), 534-539; Schaper, Circ. Res. 79 (1996), 911-919; Anderson, Science 256 (1992), 808-813; Isner, Lancet 348 (1996), 370-374; Muhlhauser, Circ. Res. 77 (1995), 1077-1086; Wang, Nature Medicine 2 (1996), 714-716; WO 94/29469; WO 97/00957 or Schaper, Current Opinion in Biotechnology 7 (1996), 653-640, and references cited therein. The DNA construct may be designed for direct introduction or for introduction via liposomes, or viral vectors (e.g. adenoviral, retroviral) into the cell and as described above. Preferably, said cell is a germ line cell, embryonic cell, or egg cell or derived therefrom, most preferably said cell is a stem cell.

Suitable gene delivery systems that can be employed in accordance with the invention may include liposomes, receptor-mediated delivery systems, naked DNA, and viral vectors such as herpes viruses, retroviruses, adenoviruses, and adeno-associated viruses, among others known in the art or described herein. Delivery of nucleic acids to a specific site in the body for gene therapy may also be accomplished using a biolistic delivery system, such as described by Williams (Proc. Natl. Acad. Sci. USA 88 (1991), 2726-2729). Standard methods for transfecting cells are well known to those skilled in the art of molecular biology, see, e.g. WO 94/29469; see also supra. Gene therapy may be carried out by directly administering the combination of DNA segments or vector of the invention ex vivo and infusing cells into the patient.

The figures show:

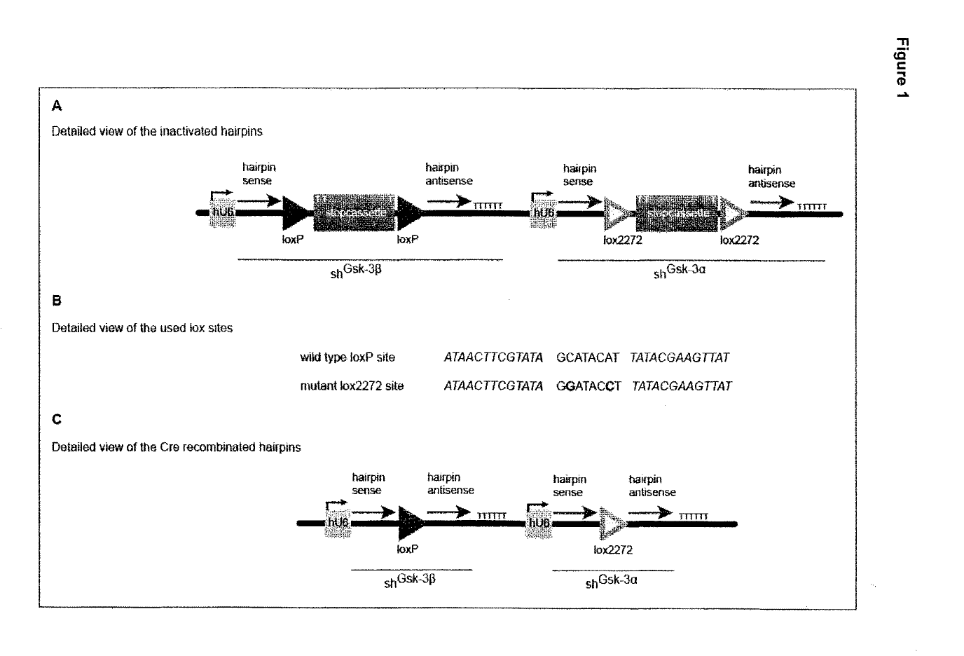

FIG. 1: Conditional short hairpin constructs

The short hairpins are driven by a human U6 promoter and are interrupted in their loop region by a stop cassette. The stop cassette consists of a 800 base pairs spacer, flanked by Cre recombinase recognition sites, so called Iox sites. Additionally, there are several stop signals situated in the stop cassette to disable the transcription completely (A). The Iox sites of the short hairpins differ in two base pairs. The inverted repeats are marked in italic, while the mutated base pairs in the Iox2272 site are marked in red (B). During the Cre recombinase mediated deletion the stop cassettes are cut out, leaving one of the Iox sites behind. The remaining Iox sites do not interfere with the activity of the short hairpins (C).

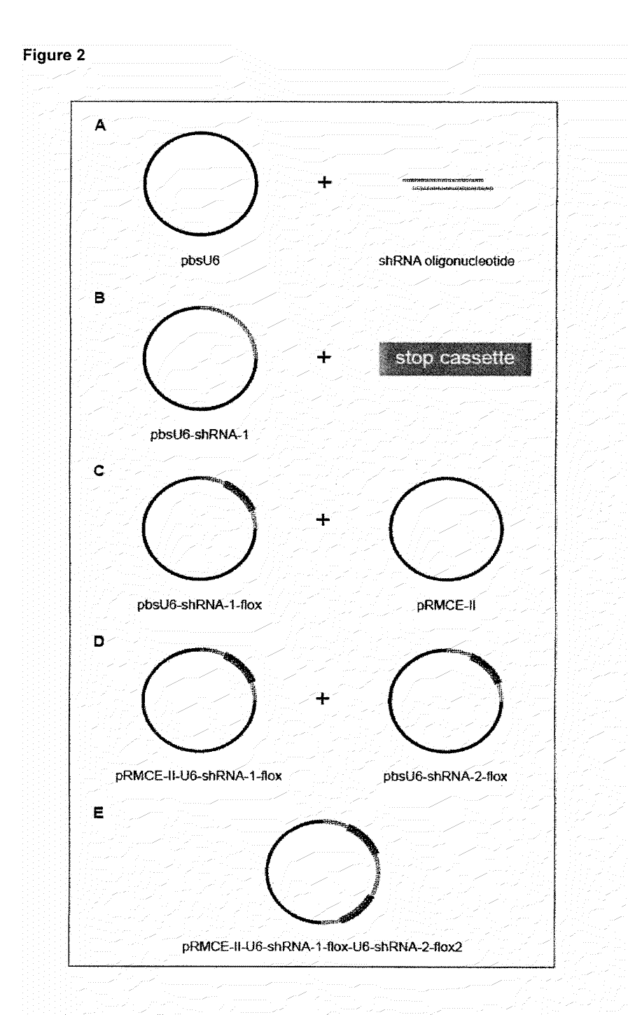

FIG. 2: Cloning Strategy

First, the short hairpin oligo-nucleotides are cloned behind a U6 promoter (A). In the next step, the stop cassette is integrated into the loop region of the hairpin (B). The conditional hairpin is transferred into an exchange vector, containing a resistance gene and recognition sites for the C31 integrase (C). The second conditional hairpin is cloned into the exchange vector behind the first construct (D). Finally, both conditional hairpin constructs, each with its independent U6 promoter, are situated in a row in the exchange vector for recombinase mediated cassette exchange (E).

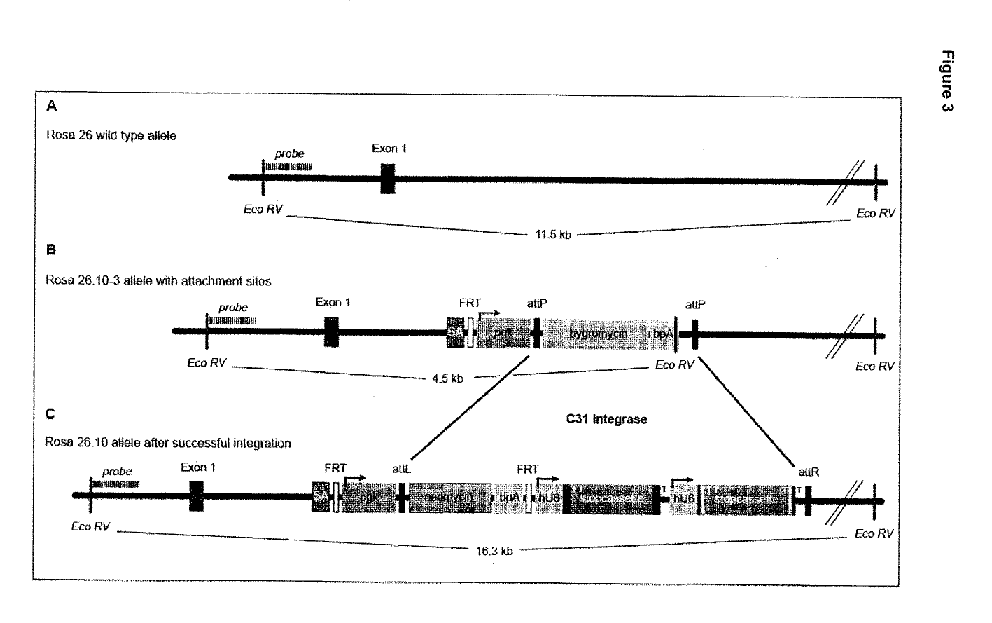

FIG. 3: Generation of recombinant ES cells

The hairpin expression cassettes are integrated in the Rosa26 locus in the mouse genome (A). The wild type locus is modified with an acceptor cassette that consists of a pair of recognition sites of the C31 Integrase, flanking a hygromycin resistance gene, driven by a pgk promoter (B). Cotransfection of the hairpin expression cassettes and a C31 Integrase expression vector into ES cells leads to the stable integration of the hairpins into the genome (C).

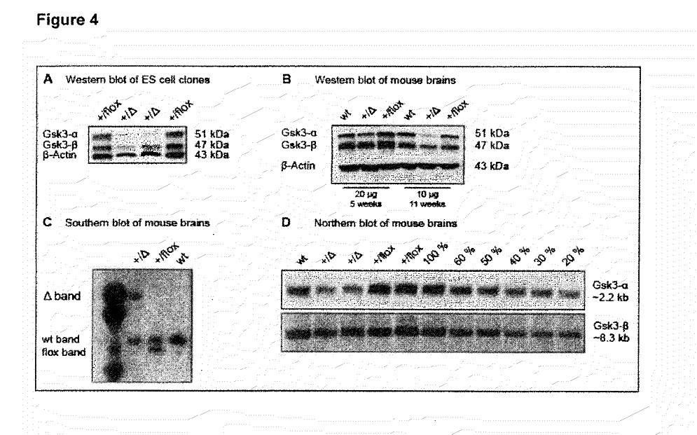

FIG. 4: Functional knockdown of Gsk-3α and Gsk-3β

As control wild type (wt) mice or heterozygous floxed (+/flox) mice were used. Mutant mice are heterozygous for the active hairpin (+/Δ) and carry a copy of the Nestin-controlled Cre recombinase.

FIG. 5: Structure of shRNA molecules

Schematic drawing of elements comprised in one of the DNA segments making up the combination according to the invention before and after a recombination event mediated by Cre recombinase and the resulting RNA transcripts.

The example illustrates the invention:

### EXAMPLE

Glycogen synthase kinase-3 (Gsk-3) is a serine-threonine kinase and was first identified as a consequence of its phosphorylation activity toward glycogen synthase, the rate limiting enzyme of glycogen metabolism. In mammals, there are two isoforms called glycogen synthase kinase 3 alpha and beta (Gsk-3α, Gsk-3β). The two genes share 85% overall sequence identity and 93% in the catalytic domain (Ali et al., Chem Rev; 101:2527-2540 (2001)). Their discrete functions are not yet elucidated, but it is known that they share some. To investigate the role of the two kinases it is necessary to eliminate them both in the same tissues and at the same time. The invention is appropriate for this question and this example shows its application in a mouse model.

A. Construction of Conditional Double Short Hairpin Vectors

To gain control over the expression of the two short hairpins, it is necessary to insert a transcriptional stop element. Therefore, the loop region of the hairpins was chosen (FIG. 1. A). The stop element consists of an 800 base pairs long spacer, containing several transcriptional stops. It is flanked by Cre recombinase recognition sites, which allow the deletion of the stop cassette by Cre recombinase. After the excision, one of the Iox sites is left behind (FIG. 1. C). It is possible to use different incompatible Iox sites, to ensure the independent control over both hairpins. The wild type IoxP site and the mutated Iox2272 site differ in two base pairs (FIG. 1. B).

The oligo-nucleotides, with the selected short hairpin sequences for Gsk-3a (SEQ ID NOs.: 4 (sense) and 5 (antisense)) and Gsk-3β (SEQ ID NOs.: 6 (sense) and 7 (antisense)), were cloned into a pbsU6 vector (SEQ ID NO.: 8). First, the oligo-nucleotides were annealed and subsequently ligated into the pbsU6 vector, digested with BseRI and BamHI (FIG. 2. A). The stop cassettes were integrated into the pre-designed HindIII site in the loop sequence of the shRNAs (FIG. 2. B). The stop cassette flanked by the wild type IoxP sites (SEQ ID NO.: 9) was integrated into the pbsU6 vector with the shRNA against Gsk-3β (SEQ ID NO.: 12; pbsU6-shGsk-3β-flox) and the stop cassette flanked by the mutated Iox2272 sites (SEQ ID NO.: 10) was integrated into the pbsU6 vector containing the shRNA against Gsk-3α (SEQ ID NO.: 11; pbsU6-shGsk-3α-flox2). To generate mice, the vector was inserted into ES cells via recombinase mediated cassette exchange (Hitz, et al., Nucleic Acids Res, 35, e90 (2007)). Therefore, the pbsU6 vectors had to be cloned into an exchange vector (SEQ ID NO.: 13: pRMCE-II) containing recognition sites (attP sites) for integrase of the phage C31. First, the pbsU6-shGsk-3β-flox vector was digested with AsiSI and Sfil to cut out the U6 promotor and the shRNA including the stop cassette. The fragment was cloned into the exchange vector, using the same restriction sites (SEQ ID NO.: 14; pRMCE-II-U6-shGsk-3β-flox, FIG. 2. C). The pbsU6-shGsk-3α-flox2 vector was digested with XbaI and the fragment was inserted into the pRMCE-II-U6-shGsk-3β-flox vector via the SpeI restriction site (SEQ ID NO.: 15: pRMCE-II-U6-shGsk-3β-flox-U6-shGsk-3α-flox2, FIG. 2. D). Thus, the Gsk-3α shRNA is situated downstream of the Gsk-3β shRNA (FIG. 2. E).

Before transfecting the vector stably into ES cells, the efficiency of the shRNA vectors can be tested in transient transfections and subsequent analysis of the protein reduction in western blot.

B. Generation and Genotyping of Recombinant ES Cells

The tested vector was electroporated into IDG26.10-3 acceptor ES cells together with a C31 integrase expression vector (SEQ ID NO.: 16: pCAG-C31Int-bpA). The acceptor ES cells contain a modified Rosa26 allele that consists of a pgk promotor and a hygromycin resistance gene, flanked by recognition sites (attB sites) for the C31 integrase (FIG. 3. B) (Hitz, et al., Nucleic Acids Res, 35, e90 (2007)). C31 integrase exchanges the sequence in the genome between the attB sites with the attP flanked sequence of the introduced vector. So, the neomycin resistance and the U6 promoter with the shRNA were integrated into the Rosa26 locus (FIG. 3. C). The ES cells were kept on MMC treated G418 resistant embryonic fibroblasts (feeder cells). Two days after the transfection, the ES cells were selected with G418 (140 μg/ml) for six days to identify recombinant clones. After this time round colonies with sharp borders were picked, separated and expanded. During this time, it is essential to prevent the differentiation of the ES cells.

To analyse the genotype of the clones, the genomic DNA of some ES cells from every expanded clone was extracted. Positive clones were analysed by PCR and Southern Blot. The integrated construct can be amplified by a primer in the pgk promoter (5′-CAC GCT TCA AAA GCG CAC GTC TG-3′; SEQ ID NO.: 28) and a reverse primer in the neomycin resistance (5′-GTT GTG CCC AGT CAT AGC CGA ATA G-3′; SEQ ID NO.: 29), giving a product of 280 base pairs at 65° C. The feeder cells, which harbour a pgk-neo resistance gene, give rise to an additional band of 160 base pairs. ES cell clones that did not undergo a complete recombination event (partial recombination, random integration, mixed clones) contain the hygromycin gene, which can be identified by a PCR, using the primers Hyg-1 (5′-GAA GAA TCT CGT GCT TTC AGC TTC GAT G-3′; SEQ ID NO.: 30) and Hyg-2 (5′-AAT GAC CGC TGT TAT GCG GCC ATT G-3′; SEQ ID NO.: 31) at a temperature of 65° C. The product of this PCR has 550 base pairs. The Rosa26 wild type allele can be identified with the primers Rosa-5′ (5′-CGT GTT CGT GCA AGT TGA GT-3′; SEQ ID NO.: 32) and Rosa-3′ (5′-ACT CCC GCC CAT CTT CTA G-3′; SEQ ID NO.: 33), giving a product of 536 base pairs at 57° C. To identify the genotype of the clones via Southern Blot, the genomic DNA was digested with the restriction enzyme EcoRV. The DNA was detected with a 448 base pairs long Rosa26 specific probe, situated upstream of the first exon and amplified with the primer pair 5′-aaggatactggggcatacg-3′ (SEQ ID NO.: 34) and 5′-cttctcagctacctttacacacc-3′ (SEQ ID NO.: 35). Recombined clones display the 11.5 kB Rosa26 wild type fragment and a 16.3 kB fragment, which derives from the correct recombined locus. An 11.4 kB fragment is detectable in case of a partial recombination and without recombination the original 4.5 kB fragment of the hygromycin-positive acceptor cells can be detected. Later, the generated mice can be genotyped by the pgk-neo PCR or by Southern Blot.

C. In Vitro Activation of the Double Hairpins in ES Cells

One of the confirmed recombinant ES cell clones was expanded and transfected with a Cre recombinase expression vector (SEQ ID NO.: 17: pCAG-Cre-bpA). The transfected cells were distributed in a very low concentration of 1,000 cells per 10 cm plate. After six days the colonies were picked, separated and expanded.

To analyse the genotype of the clones, the genomic DNA of some ES cells from every expanded clone was extracted. The clones were analysed via Southern blot, using the Rosa26 specific probe. In contrast to the protocol in section B. the genomic DNA is digested with BamHI. Cre recombinase mediated cleavage of the stop cassettes results in a 9.0 kB fragment, while the undeleted, foxed allele gives rise to a 5.4 kB fragment. A 5.8 kB fragment derives from the Rosa26 wild type allele.

D. Breeding and Probe Preparation of Double Knockdown Mice

After generating mice out of recombinant ES cell clones and germline transmission of the mutation, the mice were crossed homozygous for the foxed hairpin allele. These homozygous mice were crossed to a Cre recombinase expressing mouse strain to mediate the activation of the hairpins in the desired tissue(s). We used a mouse strain expressing Cre recombinase under the control of the rat Nestin (Nes) promotor and enhancer (Tronche et al., Nat Genet; 23:99-103 (1999)). The Nestin-cre mice express Cre recombinase in neuronal and glia cell precursors and show a strong expression of Cre recombinase in the central and peripheral nervous system. The mutant mice are heterozygous for the hairpin and Cre recombinase. At the age of three weeks, the pups were separated from their parents and genotyped. At the age of five weeks, a wild type (wt), a heterozygous floxed (+/flox), and a mutant mouse (+/Δ) were suffocated by CO₂, decapitated and the brains were rapidly dissected on ice. The brains were bisected sagittally and one half was frozen immediately on dry ice, used later for RNA extraction or storage in liquid nitrogen. The second half was stored on ice or at −20° C. until protein preparation.

E. Immunoblotting and Quantification via Northern Blotting

For protein extraction, the tissue/ES cells were homogenized in RIPA buffer (50 mM Tris-HCl pH 7.4, 1% NP-40, 0.25% sodiumdesoxycholat, 150 mM NaCl, 1 mM EDTA, protease inhibitor), sanificated, and centrifuged. Of each sample 10 and 20 μg protein were run on a 10% Tris-HCl gel (Biorad) and blotted on a PVDF membrane (Pall). After blocking with 4% skim milk the membrane was incubated with the first antibody (1 hour), washed with TBS-T, incubated with the second horseradish-peroxidase-conjugated antibody (1 hour) and washed with TBST. The detection reaction was initiated with ECL detection reagents (Amersham) and the membrane was exposed to Hyperfilm (Amersham). The antibodies used for Western blotting were anti-β-Actin (AC-15, #ab6276, Abcam, 1:100,000), anti-GSK-3α/β (#KAM-ST002, Stressgen, 1:10,000), anti-mouse (Dianova, 1:1,000).

The RNA was extracted from the tissue with TriReagent (Sigma) according to the manufacturer's protocol and solved in 100 μl Sodiumcitrate (1 mM, pH 6.4). The OD was measured and 20 μg per sample were loaded to a 1% Agarose gel. Afterwards, the RNA was transferred to a nylon membrane (Amersham) and hybridised with a gene specific probe. The radioactive labelled probe was detected with a Kodak BioMax MS film for several hours or overnight at −80° C., depending on the signal intensity. For quantification of band intensities the membrane was exposed to an Imaging Plate, from which signals were detected and scanned with the FLA-3000 imaging analyzer. After the first probe (Gsk-3α), the membrane was stripped and treated subsequently with the second gene specific probe (Gsk-3β), and at last with the loading control β-Actin.

F. Results

The conditional short hairpins, directed against Gsk-3α and Gsk-3β, are integrated in one step, at the same time, in direct neighbourhood, into the genome of mouse ES cells. Already in vitro we could achieve the proof-of-principle for this technique. We performed the Cre recombinase mediated deletion of the stop cassettes in ES cell culture by transfecting them with a Cre recombinase expression vector. A Western blot of the protein shows a clear and strong reduction of both Gsk-3 isoforms in the recombined clones (FIG. 4. A),

Additionally, we could prove the technique in vivo. We crossed the generated mice with mice expressing Cre recombinase in neuronal tissues. The brains of mutant mice (+/Δ) were used for Western blots. In FIG. 4. B, brain protein of six animals was applied with different protein amounts. The reduction in protein levels of both Gsk-3 isoforms is obvious in the +/Δ animals. To prove the total deletion of the stop cassettes, we performed a Southern blot. FIG. 4. C shows the complete deletion of the stop cassettes. The band of the floxed allele disappeared completely in the +/Δ animal. Instead, a comparably strong band for the deleted allele appeared. Besides, we measured the knockdown efficiency by Northern blotting. Again, on mRNA level we could detect a clear reduction in the +/Δ animals. For Gsk-3α the reduction is around 60%, for Gsk-3β we determined a reduction of 50% (FIG. 4. D).

1. A combination of DNA segments comprising:
(a) a first segment comprising in 5′ to 3′ or 3′ to 5′ order:
(aa) a promoter;
(ab) a first DNA sequence comprising:
(i) a DNA sequence giving rise upon transcription to the sense strand of an shRNA molecule;
(ii) a transcriptional stop element which is flanked by a first type of recombinase recognition sequences; and
(iii) a DNA sequence giving rise upon transcription to the antisense strand of an shRNA molecule;
(b) a second segment comprising in 5′ to 3′ or 3′ to 5′ order:
(ba) a promoter;
(bb) a second DNA sequence comprising:
(i) a DNA sequence giving rise upon transcription to the sense strand of an shRNA molecule;
(ii) a transcriptional stop element which is flanked by a second type of recombinase recognition sequences; and
(iii) a DNA sequence giving rise upon transcription to the antisense strand of an shRNA molecule;
wherein
(i) said first type of recombinase recognition sequences are recognized and recombined by a recombinase but not recombined with said second type of recombinase recognition sequences;
(ii) said second type of recombinase recognition sequences are recognized and recombined by the recombinase of (i) but not recombined with said first type of recombinase recognition sequences; and
(iii) said DNA sequence of (ab) and (bb) is expressed under the control of said promoters of (aa) and (ba) upon removal of said transcriptional stop elements of (ab) and (bb) by the activity of a recombinase, resulting in transcription of said shRNA molecule in a cell.

2. The combination of DNA segments of claim 1, wherein said segments are contained in the same DNA molecule.

3. The combination of claim 1 or 2, wherein said combination of DNA segments is part of a vector.

4. The combination of DNA segments of claim 1, wherein said recombinase recognition sequences are Iox sequences encompassing a wild type IoxP sequence and a mutant IoxP sequence.

5. The combination of DNA segments of claim 4, wherein said recombinase is a Cre recombinase having the sequence of SEQ ID NO.: 1.

6. The combination of DNA segments of claim 4, wherein either the first or the second type of IoxP sequences has the sequence of SEQ ID NO.: 2.

7. The combination of DNA segments of claim 4, wherein either the first or the second type of IoxP sequences has the sequence of SEQ ID NO.: 3.

8. The combination of DNA segments of claim 2, wherein the transcriptional stop element contains sequences that interfere with RNA polymerase III driven transcription.

9. The combination of DNA segments of claim 1, wherein the promoters are promoters of genes transcribed by RNA polymerase III.

10. The combination of DNA segments of claim 9, wherein the promoters are selected from the group consisting of U6 or H1 gene promoters.

11. The combination of DNA segments of claim 2, wherein said DNA molecule containing said combination of DNA segments comprises further elements allowing for stable integration of said molecule into the genome of a non-human animal.

12. The combination of DNA segments of claim 11, wherein said further elements allow for site-specific integration.

13. The combination of DNA segments of claim 11, wherein the further elements for stable integration into the genome are sequences suitable for integration of the DNA molecule containing said combination of DNA segments through recombination.

14. The combination of DNA segments of claim 11, wherein the integration site is a genetic locus comprising sequences suitable for integration of the DNA molecule containing said combination of DNA segments molecule through recombination.

15. The combination of DNA segments of claim 13 or 14, wherein sequences suitable for integration through recombination are recognition sites for enzymes mediating recombination events.

16. The combination of DNA segments of claim 15, wherein the enzymes mediating recombination events are DNA recombinases or integrates.

17. The combination of DNA segments of claim 11, wherein said DNA molecule containing said combination of DNA segments is integrated into a genetic locus having a mild or ubiquitous transcriptional activity.

18. The combination of DNA segments of claim 11, wherein said DNA molecule containing said combination of DNA segments is integrated at the Rosa26 locus or Hypoxanthin-Phosphoribosyl-Transferase (HPRT) locus.

19. A method of producing a transgenic non-human animal, the method comprising the steps of (a) integrating the DNA molecule containing said combination of DNA segments of claim 2 into the genome of a non-human animal; and of (b) crossing said animal with an animal transgenic for an expressible recombinase gene, wherein said recombinase recognizes the recombinase recognition sequences flanking the transcriptional stop element of said DNA segments.

20. A genetically engineered non-human animal transgenic for the DNA molecule containing said combination of DNA segments of claim 2 and an expressible recombinase gene.

21. The genetically engineered non-human animal of claim 20, wherein the recombinase is expressed under the control of a tissue-specific promoter.

22. The genetically engineered non-human animal of claim 20, wherein the non-human animal is a rodent.

23. The genetically engineered non-human animal of claim 22, wherein the rodent is a mouse.

24. A eukaryotic cell genetically engineered with the combination of DNA segments of claim 1.

25. A method of simultaneously knocking down two genes in a eukaryotic cell comprising the steps of:
(a) introducing the combination of DNA segments of claim 1 into a cell;
(b) excising the transcription stop elements of (ab) and (bb) through the activity of a recombinase.

26. The eukaryotic cell of claim 24, wherein the recombinase is expressed in said cell.

27. The eukaryotic cell of claim 26, wherein the recombinase is expressed under the control of a tissue-specific promoter.

28. The method of claim 25, wherein the recombinase is exogenously introduced into the cell.

29. A method of identifying a combination of two target genes as a potential drug target comprising the steps of:
(a) determining different expression or activity of nucleic acid molecules or proteins in a cell exhibiting characteristics associated with a disease and in a normal cell;
(b) knockdown of two genes according to the method of claim 25 in said cell exhibiting characteristics associated with a disease; and
(c) determining the effect of the knockdown on said cell exhibiting characteristics associated with a disease; wherein a change in said disease characteristics is indicative that said combination of two target genes is a potential drug target.

30. The method of claim 29, wherein at least one of the genes is known to be associated with said disease.

31. The method of claim 29, wherein the cell exhibiting characteristics associated with a disease is obtained from a patient.

32. The method of claim 29, wherein steps (a) to (c) are performed in a mouse.

33. The combination of DNA segments of claim 1, wherein the cell is a eukaryotic cell.

34. The cell of claim 24 or the method of claim 25, wherein said eukaryotic cell is selected from the group consisting of a non-human embryonic stem cell, a cell contained in a tissue sample and a cell contained in a transgenic non-human mammal.

35. A pharmaceutical composition comprising the combination of DNA segments of claim 1.

36.-37. (canceled)
